(19) United States
(12) Patent Application Publication
(10) Pub. No.: US 2007/0243548 A1
(43) Pub. Date: Oct. 18, 2007

(54) Calumenin-directed diagnostics and therapeutics for cancer and chemotherapeutic drug resistance

(76) Inventors: Elias Georges, Laval (CA); Panagiotis Prinos, Montreal (CA)

Correspondence Address: WILMER CUTLER PICKERING HALE AND DORR LLP, 60 STATE STREET, BOSTON, MA, 02109, US

(21) Appl. No.: 11/729,298

(22) Filed: Mar. 28, 2007

Related U.S. Application Data
(60) Provisional application No. 60/786,745, filed on Mar. 28, 2006.

(51) Int. Cl. C12Q 1/68; G01N 33/574

(52) U.S. Cl. 435/6; 435/7.23

(57) ABSTRACT

Methods are disclosed for diagnosing chemotherapeutic drug resistance in neoplastic cells by detecting an increase in the expression of calumenin in such neoplastic cells as compared to the level of expression of calumenin protein in a non-MDR neoplastic cell. In addition, disclosed are methods for treating neoplastic cells, including reversing or preventing chemotherapeutic drug resistance, by increasing the sensitivity of the neoplastic cells to a chemotherapeutic drug.

### CROSS REFERENCE TO RELATED APPLICATIONS

This application claims the benefit of U.S. Provisional Application No. 60/786,745 filed Mar. 28, 2006, entitled “Calumenin-Directed Diagnostics and Therapeutics for Cancer and Chemotherapeutic Drug Resistance,” the contents of which are hereby incorporated by reference in its entirety.

### FIELD OF THE INVENTION

This invention relates to the field of cancer. In particular, this invention relates to the detection, diagnosis, and treatment of neoplastic cells, and more specifically to the detection and treatment of chemotherapeutic drug-resistant neoplastic cells.

### BACKGROUND OF THE INVENTION

Diseases such as cancer are typically treated with chemotherapeutics such as cytotoxic drugs. In order to kill the cancer or diseased cells, the drug(s) must enter the cells and reach an effective dose so as to interfere with essential biochemical pathways. Generally, chemotherapeutic drugs disrupt cellular mechanisms such as DNA replication and osmotic control to bring about apoptosis of the cell. Although chemotherapeutic drugs are effective at killing neoplastic cells, they also tend to be indiscriminate killers of other cells in the subject, targeting healthy and neoplastic cells with equal efficacy. As a result, chemotherapy treatments are generally provided to the subject for as short of a period as possible to limit the detrimental effects of the drug on the subject.

Chemotherapy drug treatments can be limited by the inherent sensitivity of the cancer cell to the drug being used in the treatment, which can vary from cancer type to cancer type. In some cases, a treatment regime lasting for a long duration can be required due to the relative insensitivity of the cells to the treatment, increasing the patient's exposure to drugs that are toxic to both normal and cancer cells. However, as described above, prolonged treatment periods can increase the likelihood that the patient will suffer from detrimental side effects attributable to the treatment regime. Common side effects include neutropenia, anemia, thrombocytopenia, nausea, hair loss, organ and tissue damage, and infections. Although most side effects are normally tolerable compared to the symptoms of the disease, chemotherapeutic side effects can, in some instances, lead to cessation of the treatment regime or death. As a result of these potentialities, many patients suffer significant emotional and physiological consequences associated with the treatment regime.

In addition to the inherent sensitivity of particular cancer cell types to chemotherapeutic drugs, cancer cells can evade being killed by the drug through the development of resistance to it (termed “drug resistance”). Moreover, in some cases, cancer cells (also called tumor cells or neoplastic cells) develop resistance to a broad spectrum of drugs, including drugs that were not originally used for treatment. This phenomenon is termed “chemotherapeutic drug resistance.” Chemotherapeutic drug resistance arises through different mechanisms, and each mechanism is associated with a different biological marker or group of markers that can be clinically useful for detecting and diagnosing the presence of drug resistance.

The emergence of the chemotherapeutic drug resistance, and also the multi-drug resistance (“MDR”) phenotype is the major cause of failure in the treatment of cancer (see, e.g., Davies (1994) Science 264: 375-382; Poole (2001) Cur. Opin. Microbiol. 4: 500-5008). The chemotherapeutic drug resistance phenotype can arise in response to a broad spectrum of functionally distinct drugs, whereby treatment options are significantly limited by chemotherapeutic drug resistance development. The development of chemotherapeutic drug-resistant cancer cells is therefore the principal reason for treatment failure in cancer patients (see, e.g., Gottesman (2000) Ann. Rev. Med. 53: 615-627).

The sensitivity of cancer cells to a particular drug is normally associated with genes that are utilized in drug metabolism or transport (see, e.g., Volm et al., (1993) Cancer 71: 3981-3987). For example, the classic multi-drug resistance phenotype involves alterations in a gene for P-glycoprotein, a plasma membrane protein that actively transports drugs out of the cell (see id.). In addition to the P-glycoprotein gene, there are many genes that affect the sensitivity of a cancer cell to a particular drug or class of drugs (see, e.g., Di Nicolantonio et al., (2005) BMC Cancer. 5(1): 78). Thus, it is clear that chemotherapeutic drug sensitivity and multi-drug resistance are multi-factorial traits.

One class of proteins that has been associated with chemotherapeutic drug resistance is the calcium-binding proteins (see, e.g., Hegde et al., (2004) Eur. J. Med. Chem. 39(2): 161-77). Calcium-binding proteins such as calmodulin modulate cell-signaling machinery and associate with a diverse range of proteins. Calcium-binding proteins have also been associated with cell cycle regulation and regulation of cell death. Therefore, calcium-binding proteins appear to be important regulators of cellular functions that have been identified as potential targets for cancer therapy.

Recently, several groups have identified calumenin, a novel calcium-binding protein, as an important component of the Vitamin K γ-carboxylase system (see, e.g., Yabe et al. (1997) J. Biol. Chem. 272(29): 18232-18239). Calumenin associates with the vitamin K 2,3-epoxide reductase (VKOR) protein complex in the ER membrane and the Golgi complex (see, e.g., Yabe et al. (1997) J. Biol. Chem. 272(29): 18232-18239). Furthermore, calumenin has been found in most cells of the body, and has been identified as a protein that is exocytosed from cells such as fibroblasts and keratinocytes (Vorum et al. (1999) Exp. Cell Res. 248(2): 473-481; Coppinger et al. (2004) Blood. 103(6): 2096-2104). In addition, decreased calumenin expression has been associated with increased resistance to platinum containing drugs in certain cell lines (Kim et al. (2003) Cancer Ther. 1: 9-20).

There remains a need in both humans and animals for methods and compositions that detect, treat, and prevent cancer. Furthermore, there remains a need in both humans and animals for detecting, treating, preventing, and reversing the development of both classical and atypical MDR phenotypes in cancer cells and non-cancerous damaged cells, regardless of how the MDR arises (e.g., naturally occurring or drug-induced). In addition, the ability to identify, and to make use of, reagents that target cancer cells and multiple drug-resistant cells has clinical potential for improvements in the diagnosis of chemotherapeutic drug resistance. Such reagents also have the clinical potential to improve treatments for cancer, including chemotherapeutic drug-resistant cancers. Also, there remains a need in both humans and animals for increasing the sensitivity of cancer cells to chemotherapeutic drugs in order to shorten the time period of chemotherapeutic treatment. By shortening the time period of chemotherapeutic treatment and allowing physicians to make appropriate chemotherapy treatment choices, there is a potential for significant improvements in treatment of neoplasms.

### SUMMARY OF THE INVENTION

The present invention is based, in part, upon the discovery that calumenin, a calcium binding protein localized to the endoplasmic reticulum and the Golgi complex of the cell, is expressed at higher levels in neoplastic cells that have developed chemotherapeutic drug resistance. This discovery has been utilized to provide the present invention that, in part, is directed to therapeutic methods and compositions for treating neoplastic cells, including neoplastic cells that have developed chemotherapeutic drug resistance, through the use of targeting agents specific for calumenin. Moreover, calumenin expression levels are diagnostic of chemotherapeutic drug resistance. The invention, in part, also provides a method that uses targeting agents specific for calumenin to detect and diagnose chemotherapeutic drug resistance in neoplastic cells in a subject.

Accordingly, one aspect of the invention provides a method for diagnosing chemotherapeutic drug resistance in a neoplastic cell. The method comprises the detection of a level of calumenin expressed in a neoplastic cell sample, and also the detection of a level of calumenin in a non-resistant neoplastic cell of the same tissue type as the neoplastic cell sample. The method entails comparing the level of calumenin expressed in the neoplastic cell sample to the level of calumenin expressed in the non-resistant neoplastic cell of the same tissue type or origin. Chemotherapeutic drug resistance is indicated if the level of calumenin expressed in the neoplastic cell sample is greater than the level of calumenin expressed in the non-resistant neoplastic cell of the same tissue type or origin.

In another aspect, the invention provides a method of diagnosing chemotherapeutic drug resistance in a neoplastic cell. The method comprises detecting a level of calumenin expressed in a neoplastic cell sample by contacting the cell sample with a probe specific for calumenin. The neoplastic cell is not obtained, or derived from, a cervix squamous cell carcinoma. The method entails detecting a level of calumenin expressed in a non-resistant neoplastic cell control sample of the same tissue type as the neoplastic cell sample by contacting the cell sample with a calumenin-specific probe. The level of expressed calumenin in the neoplastic cell sample is compared to a level of expressed calumenin in the non-resistant neoplastic cell. The chemotherapeutic drug-resistance is indicated in the neoplastic cell sample if the level of calumenin expressed in the neoplastic cell sample is greater than the level of calumenin expressed in the non-resistant neoplastic control cell sample.

In certain embodiments, the detection steps comprise isolating a cytoplasmic sample from the neoplastic cell sample and the non-resistant neoplastic control cell sample. In other embodiments, detecting the level of expressed calumenin in the cell samples comprises contacting the cell samples with a calumenin targeting agent selected from the group consisting of ligands, synthetic small molecules, nucleic acids, peptidomimetic compounds, inhibitors, peptides, proteins, and antibodies. In particular embodiments, the calumenin-targeting agent comprises an anti-calumenin antibody or a calumenin-binding fragment thereof. In more particular embodiments, the level of antibody bound to calumenin is detected by immunofluorescence, radiolabel, or chemiluminescence.

In certain embodiments, the detecting steps comprise hybridizing a nucleic acid probe to a complementary calumenin mRNA. In other embodiments, the nucleic acid probe is selected from the group consisting of RNA, DNA, RNA-DNA hybrids, and siRNA. In still other embodiments, the level of nucleic acid probe hybridized to calumenin mRNA is detected with a label selected from the group consisting of fluorophores, chemical dyes, radiolabels, chemiluminescent compounds, colorimetric enzymatic reactions, chemiluminescent enzymatic reactions, magnetic compounds, and paramagnetic compounds.

In certain embodiments, the neoplastic control cell sample is selected from the group consisting of lung carcinoma, lung adenocarcinoma, colon carcinoma, ovarian carcinoma, and ovarian adenocarcinoma. In particular embodiments, the neoplastic cell sample to be tested comprises a breast adenocarcinoma. In other embodiments, the neoplastic cell sample to be tested is isolated from a mammal. In yet other embodiments, the neoplastic cell sample to be tested is isolated from a human. In still other embodiments, the potentially chemotherapeutic drug-resistant neoplastic cell sample is isolated from a tissue selected from the group consisting of breast, skin, lymphatic, prostate, bone, blood, brain, liver, thymus, kidney, lung, and ovary.

In still another aspect, the invention provides a method of treating a neoplasm that is not, or is not derived from, a cervix squamous cell carcinoma in a patient in need thereof. The method comprises administering an effective amount of a calumenin-targeting agent to the patient, the targeting agent being capable of binding to calumenin expressed in the neoplasm. The method further entails administering to the patient an effective amount of a chemotherapeutic drug. The calumenin-targeting agent, when bound to the neoplasm, increases the sensitivity of the neoplasm to the chemotherapeutic drug.

In certain embodiments, the calumenin-targeting agent bound to the neoplasm is internalized into the neoplastic cell. In other embodiments, the calumenin-targeting agent comprises a liposome. In still other embodiments, the liposome comprises a neoplastic cell-targeting agent on its surface.

In certain embodiments, the calumenin-targeting agent is selected from the group consisting of ligands, nucleic acids, synthetic small molecules, peptidomimetic compounds, inhibitors, peptides, proteins, and antibodies. In particular embodiments, the calumenin-targeting agent comprises a nucleic acid. In more particular embodiments, the nucleic acid is complementary to a calumenin mRNA. In still more particular embodiments, the nucleic acid is selected from the group consisting of RNA, DNA, RNA-DNA hybrids, and siRNA. In yet more particular embodiments, the siRNA comprises 19 contiguous nucleotides of SEQ ID NO: 2 or it comprises 25 contiguous nucleotides of SEQ ID NO: 4.

In certain embodiments, the calumenin-targeting agent comprises an antibody or calumenin-binding fragment thereof. In other embodiments, the neoplastic cell-targeting agent comprises an antibody, or antigen-binding fragment thereof, specific for a cell marker selected from the group consisting of multidrug resistance protein 1, BRCP, p53, vimentin, α-enolase, nucleophosmin, and HSC70.

In certain embodiments, the calumenin-targeting agent is administered to the patient by injection at the site of the neoplasm. In other embodiments, the calumenin-targeting agent is administered to the patient by surgical introduction at the site of the neoplasm. In still other embodiments, the calumenin-targeting agent is administered to the patient by inhalation of an aerosol or vapor.

In certain embodiments, the neoplasm to be treated is chemotherapeutic drug-resistant. In particular embodiments, the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristine, Vinorelbine, and combinations thereof.

In another aspect, the invention provides a kit for detecting chemotherapeutic drug resistance in a neoplastic cell sample. The kit comprises a first probe for the detection of calumenin and a second probe for the detection of chemotherapeutic drug resistance, the second probe being specific for a marker selected from the group consisting of multidrug resistance protein 1, BRCP, p53, vimentin, α-enolase, nucleophosmin, and HSC70. The kit provides at least one detection means for identifying probe binding to a target.

In certain embodiments, the first probe is selected from the group consisting of ligands, nucleic acids, synthetic small molecules, peptidomimetic compounds, inhibitors, peptides, proteins, and antibodies. In other embodiments, the first probe is a nucleic acid that is complementary to mRNA encoding calumenin. In particular embodiments, the nucleic acid is selected from the group consisting of RNA, DNA, RNA-DNA hybrids, and siRNA.

In certain embodiments, the first probe is a calumenin-specific antibody or binding fragment thereof. In other embodiments, the second probe comprises a nucleic acid complementary to an mRNA encoding multidrug resistance protein 1, BRCP, p53, vimentin, α-enolase, nucleophosmin, or HSC70. In particular embodiments, the nucleic acid probe is selected from the group consisting of RNA, DNA, RNA-DNA hybrids, and siRNA.

In certain embodiments, the second probe comprises an antibody or calumenin-binding fragment thereof. In other embodiments, the detection means is selected from the group consisting of fluorophores, chemical dyes, radiolabels, chemiluminescent compounds, colorimetric enzymatic reactions, chemiluminescent enzymatic reactions, magnetic compounds, and paramagnetic compounds.

In yet another aspect, the invention provides a pharmaceutical formulation for treating a neoplasm. The pharmaceutical formulation comprises a calumenin-targeting component, a chemotherapeutic drug, and a pharmaceutically acceptable carrier.

In certain embodiments, the calumenin-specific targeting component is selected from the group consisting of ligands, nucleic acids, synthetic small molecules, peptidomimetic compounds, inhibitors, peptides, proteins, and antibodies. In particular embodiments, the calumenin-targeting component is a nucleic acid. In more particular embodiments, the nucleic acid is selected from the group consisting of RNA, DNA, RNA-DNA hybrids, and siRNA. In still more particular embodiments, the calumenin-targeting component is a siRNA. In certain embodiments, the siRNA has a GC content of at least 40%. In particular embodiments, the siRNA comprises 19 contiguous nucleotides of SEQ ID NO: 2. In still more particular embodiments, the siRNA comprises 25 contiguous nucleotides of SEQ ID NO: 4.

In certain embodiments, the calumenin-targeting agent comprises an antibody or calumenin-binding fragment thereof. In other embodiments, the calumenin-targeting agent comprises a liposome. In still other embodiments, the liposome comprises a neoplastic cell-targeting agent on its surface.

In certain embodiments, the neoplastic cell-targeting agent is an antibody, or binding fragment thereof. In other embodiments, the neoplastic cell-targeting agent binds to a neoplastic cell marker selected from the group consisting of multidrug resistance protein 1, BRCP, p53, vimentin, α-enolase, nucleophosmin, and HSC70. In yet other embodiments, the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristine, Vinorelbine, and combinations thereof.

### BRIEF DESCRIPTION OF THE FIGURES

The foregoing and other objects of the present invention, the various features thereof, as well as the invention itself can be more fully understood from the following description, when read together with the accompanying drawings in which:

FIG. 1A is a photographic representation of an immunoblot probed with anti-calumenin antibody that shows the level of expression of calumenin in drug-resistant (e.g., MCF-7/AR and MCF-7VLB) and drug-sensitive (e.g., MCF-7) MCF-7 cell extracts.

FIG. 1B is a photographic representation of an immunoblot probed with anti-calumenin antibody that shows the level of expression of calumenin in drug-resistant (e.g., MDA/AR and MDA/taxol) and drug-sensitive (e.g., MDA) MDA cell extracts.

FIG. 2 is a photographic representation of an immunoblot probed with anti-calumenin antibody that shows the level of expression of calumenin in cell extracts from MCF-7 cells treated with mock siRNA, siGLO control siRNA, or calumenin siRNA.

FIG. 3A is a photographic representation of a phase contrast image of a viability assay showing the effects of calumenin silencing on the viability of MCF-7 cells treated with control siRNA.

FIG. 3B is a photographic representation of a phase contrast image of a viability assay showing the effects of calumenin silencing on the viability of MCF-7 cells treated with calumenin siRNA.

FIG. 4A is a photographic representation of the results of an apoptosis assay after Annexin V staining that shows MCF-7 cells treated with control cells treated with control siRNA.

FIG. 4B is a photographic representation of the results of an apoptosis assay after Annexin V staining that shows MCF-7 cells treated with calumenin siRNA.

FIG. 5 is a graphic representation of the results of an MTT cytotoxicity assay that shows the viability of calumenin-depleted MCF-7 cells compared to mock and siGLO controls.

FIG. 6A is a graphic representation of the results of an MTT cytotoxicity assay that shows the viability of calumenin siRNA transfected MCF-7 cells treated with adriamycin (Doxorubicin) compared to mock transfected MCF-7 controls.

FIG. 6B is a graphic representation of the results of an MTT cytotoxicity assay that shows the viability of calumenin siRNA transfected MCF-7 cells treated with mitoxantrone compared to mock transfected MCF-7 controls.

FIG. 6C is a graphic representation of the results of an MTT cytotoxicity assay that shows the viability of calumenin siRNA transfected MCF-7 cells treated with vincristine compared to mock transfected MCF-7 controls.

FIG. 6D is a graphic representation of the results of an MTT cytotoxicity assay that shows the viability of calumenin siRNA transfected MCF-7 cells treated with cisplatin compared to mock transfected MCF-7 controls.

FIG. 7 is a graphic representation of the results of a clonogenic assay that shows the viability of calumenin-depleted MCF-7 cells challenged with different concentrations of taxol or vincristine (e.g., IC10 and IC50) compared to MCF-7 cells treated with control siRNA.

FIG. 8 is a graphic representation of the results of a microarray assay that shows the expression levels of calumenin in drug-resistant cell lines compared to control drug-sensitive cell lines.

### DETAILED DESCRIPTION OF THE INVENTION

The patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

1.1 General

Aspects of the present invention provide methods for diagnosing chemotherapeutic drug-resistance in a neoplastic cell. One method of the present invention includes measuring a level of expression of calumenin in a neoplastic cell sample and comparing the level of expression of calumenin in the neoplastic cell sample to the level of expression of calumenin in a non-resistant neoplastic cell of the same tissue type. If the level of expression of calumenin is greater in the neoplastic cell sample than in the non-resistant neoplastic cell, chemotherapeutic drug-resistance is indicated. In some embodiments, the neoplastic cell sample and the non-resistant neoplastic cell are separated into fractions, and the cytoplasmic fractions are tested for calumenin expression.

Furthermore, aspects of the invention also provide methods and reagents to treat and/or prevent the progression of cancer in a patient by increasing the sensitivity of the cancer cells to the chemotherapeutic drug(s). Additionally, aspects of the invention allow for the improved clinical identification and treatment of patients having chemotherapeutic drug-resistant neoplasms.

Accordingly, the present invention provides, in part, methods of treating and/or preventing cancer in a patient by increasing the sensitivity of the cancer cells to a chemotherapeutic treatment regime. The methods include administering an effective amount of a calumenin-targeting agent to a cancer patient such that the calumenin-targeting agent binds to calumenin expressed in the neoplastic cells. The patient is treated with a chemotherapeutic drug either simultaneously or subsequent to the administration of the calumenin-targeting agent to the patient.

As used herein, a “neoplastic cell” is a cell that shows aberrant cell growth, such as increased, uncontrolled cell growth. A neoplastic cell can be a hyperplastic cell, a cell from a cell line that shows a lack of contact inhibition when grown in vitro, a tumor cell when grown in vivo, or a cancer cell that is capable of metastasis in vivo. Alternatively, a neoplastic cell can be termed a “cancer cell.” Non-limiting examples of cancer cells include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, thymoma, lymphoma cells, melanoma cells, sarcoma cells, leukemia cells, retinoblastoma cells, hepatoma cells, myeloma cells, glioma cells, mesothelioma cells, and carcinoma cells. However, squamous carcinomas of or derived from cervical origin and squamous carcinoma cells of cervical origin are not included within the scope of the detection and diagnosis embodiments of the present invention.

Cancer cells can be obtained from non-limiting tissues such as breast, lung, bone, blood, skin, brain, gastrointestinal, lymphatic, hepatic, muscle, ovary, uterine, and kidney. Cancer cells can be obtained from tissues other than the tissue from which the cancer cell originally developed, as in the case of metastasized cancer cells. In the case of detection and diagnosis, cancer cells are not derived from the cervix squamous cells, and do not derive originally from cervix squamous cells. Moreover, cancer cells can be obtained from mammals including, but not limited to, human, non-human primates such as chimpanzee, mouse, rat, guinea pig, chinchilla, rabbit, pig, and sheep.

Alternatively, cancer cells can be obtained in the form of a cell line. The term “cell line,” as used herein, means any cell that has been isolated from the tissue of a host organism and propagated by artificial means outside of the host organism. Such cell lines can be chemotherapeutic drug-resistant or chemotherapeutic drug-sensitive. A cell line is isolated, or derived from, tissues such as prostatic tissue, bone tissue, blood, brain tissue, lung tissue, ovarian tissue, epithelial tissue, breast tissue, and muscle tissue. A cell line can be derived, produced, or isolated from a cancer cell type, e.g., melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, or thymoma. However, cell lines derived or originating from cervix squamous cell carcinomas are not within the scope of the diagnostic or detection embodiments of the present invention. Cell lines can also be generated by techniques well known in the art (see, e.g., Griffin et al., (1984) Nature 309(5963): 78-82). Useful, exemplary, and non-limiting cell lines include MCF7, MDA, SKOV3, OVCAR3, 2008, PC3, T84, HCT-116, H69, H460, HeLa, and MOLT4.

As used herein, “chemotherapeutic drug” means a pharmaceutical compound that kills a damaged cell such as a cancer cell. Cell death can be induced by the chemotherapeutic drug through a variety of means including, but not limited to, apoptosis, osmolysis, electrolyte efflux, electrolyte influx, cell membrane permeabilization, and DNA fragmentation. Exemplary non-limiting chemotherapeutic drugs are adriamycin, cisplatin, taxol, melphalan, daunorubicin, dactinomycin, bleomycin, fluorouracil, teniposide, vinblastine, vincristine, methotrexate, mitomycin, docetaxel, chlorambucil, carmustine, mitoxantrone, and paclitaxel.

As used herein, the term “chemotherapeutic drug-resistance” encompasses the development of resistance to a particular chemotherapeutic drug, class of chemotherapeutic drugs or multiple chemotherapeutic drugs by a cancer cell. Resistance can occur before or after treatment with a chemotherapy regime. Without being limited to any one theory, the mechanism of development of chemotherapeutic drug resistance can occur by any means, such as by pathogenic means such as through infections, particularly viral infection. Alternatively, chemotherapeutic drug resistance can be conferred by a mutation or mutations in one or several genes located either chromosomally or extrachromosomally. In addition, chemotherapeutic drug resistance can be conferred by selection of a certain phenotype by exposure to the chemotherapeutic drug or class of chemotherapeutic drugs, and then subsequent survival of the cell to the particular treatment. The above-mentioned mechanisms of chemotherapeutic drug resistance are known in the art. The terms, “chemotherapeutic drug-resistant” and “chemotherapeutic drug resistance,” are used to describe a neoplastic cell or a damaged cell that is chemotherapeutic drug-resistant due to either the classical mechanism (i.e., involving P-glycoprotein or another MDR protein) or an atypical mechanism (non-classical mechanism) that does not involve P-glycoprotein (e.g., an atypical mechanism that involves the MRP1 chemotherapeutic drug resistance marker).

As used herein, the term “MDR protein” includes any of several integral transmembrane glycoproteins of the ABC type that are involved in (multiple) drug resistance. These include MDR 1 (P-glycoprotein or P-glycoprotein 1), an energy-dependent efflux pump responsible for decreased drug accumulation in chemotherapeutic drug-resistant cells. Examples of MDR 1 include human MDR 1 (see, e.g., database code MDR1_HUMAN, GenBank Accession No. P08183, 1280 amino acids (141.34 kD)). Other MDR proteins include MDR 3 (or P-glycoprotein 3), which is an energy-dependent efflux pump that causes decreased drug accumulation but is not capable of conferring drug resistance by itself. Examples of MDR 3 include human MDR 3 (see, e.g., database code MDR3_HUMAN, GenBank Accession No. P21439, 1279 amino acids (140.52 kD). Other MDR-associated proteins participate in the active transport of drugs into subcellular organelles. Examples from human include MRP 1, Chemotherapeutic Drug Resistance-associated Protein 1, database code MRP_HUMAN, GenBank Accession No. P33527, 1531 amino acids (171.47 kD).

In some embodiments of the invention, targeting agents are used to detect the level of expression of calumenin in a cell sample. As used herein, the term “targeting agent” means a compound that can bind, associate, or hybridize with a target molecule in a specific manner. The mechanisms of binding to a target molecule include, e.g., hydrogen bonding, Van der Waals attractions, covalent bonding, ionic bonding, or hydrophobic interactions. In certain embodiments, a targeting agent is used to detect the level of expression of calumenin in a neoplastic cell sample. Non-limiting examples of targeting agents include antibodies, antibody fragments, nucleic acids, proteins, peptides, and peptidomimetic compounds.

As used herein, the term “calumenin-targeting agent” refers to compounds that can specifically bind to calumenin expressed in the cell. Calumenin can be expressed as a nucleic acid such as messenger RNA (“mRNA”) that encodes for calumenin polypeptide or a fragment of the polypeptide. Also, calumenin can be expressed as a polypeptide or as fragments of the completed polypeptide. Targeting agents include, but are not limited to, compounds such as antibodies or fragments thereof, peptides, peptidomimetic compounds, nucleic acids, and small molecules.

The present invention provides methods of detecting chemotherapeutic drug resistance in a patient. The methods include administering to a cancer patient a calumenin-targeting agent and detecting the calumenin-targeting agent that is bound to expressed calumenin using a detectable label operably linked to the calumenin-targeting agent.

Aspects of the present invention also allow the identification of those patients whose neoplastic cells have acquired chemotherapeutic drug resistance. In some situations, the patient is identified when he/she no longer responds to the drug being used in his/her treatment. For example, a breast cancer patient in remission being treated with a chemotherapeutic agent (e.g., vincristine) can suddenly come out of remission, despite being constantly treated with the chemotherapeutic agent. Unfortunately, such a patient is often found also to be unresponsive to other chemotherapeutic agents, including some to which the patient has never been exposed. Of course, after these patients become chemotherapeutic drug-resistant, treating these patients to control their now-resurgent cancer or disease caused by a damaged cell is difficult and can require more drastic therapies, such as radiotherapy or surgery (e.g., bone marrow transplantation or amputation of necrotic tissue).

Some aspects of the present invention also allow an early diagnosis of chemotherapeutic drug resistance by detecting increased amounts of calumenin in neoplastic cells of the patient. Such an early diagnosis allows patients who are initially drug responders and sensitive to drug treatment to be distinguished from those who are initially drug non-responders. Further, diagnostic procedures using calumenin expression can also be used to follow the development and emergence of MDR neoplastic cells that are resistant to the treatment drug and that arise during the course of drug treatment, permitting health professionals to tailor their treatments accordingly.

The invention also provides methods of treating or preventing the growth of resistant or chemosensitive neoplasms in a patient. The methods include administering an effective amount of calumenin-targeting agent to a patient, the targeting agent being targeted to the neoplasm or to a site in close proximity to the neoplasm. Treatment of the patient includes administering a chemotherapeutic drug to kill the neoplastic cells after the cells have been targeted by the calumenin-targeting agent to increase the chemosensitivity of the neoplastic cells to the chemotherapeutic drug. Alternatively, the targeting agent and the chemotherapeutic drug can be administered simultaneously, e.g., as a single, linked therapeutic.

The calumenin-targeting agent can be composed of multiple parts, herein termed “components.” For example, the calumenin-targeting agent can have a cell-associating component. A useful cell-associating component is an antibody or binding fragment of an antibody such as Fv, F(ab′)₂, F(ab), Dab, and SC-Mab that binds to cell surface expressed cancer cell markers such as Pgp-1, MRP1, BIP, BRCP, HSC70, nucleophosmin, vimentin, and HSP90. The cell-associating component can also be a compound that binds to a cell marker such as, but not limited to, an inhibitor of a cancer cell marker, a peptide, a peptidomimetic, a ligand, or a small molecule. As used herein, the term “inhibitor” means a compound that prevents a biomolecule, e.g., a protein, nucleic acid, or ribozyme, from completing a reaction. An inhibitor can inhibit a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme.

As long as the interaction of the cell-associating component allows for cancer cell-specific targeting of the calumenin-targeting agent, a compound is useful as a cell-associating component. The calumenin-targeting agent also can include a cell-internalization component that allows the calumenin-targeting agent to enter into the cell. For example, a cell-internalization component can be an agent that allows for cell membrane fusion between the calumenin-targeting agent and the cancer cell, such as a liposome or immunoliposome (see, e.g., Drummond, et al, (2005) Ann. Rev. Pharmacol. Toxicol. 45: 495-528).

The cell-internalization component can be a dendrimer conjugate, which is a spherical polymer (see, e.g., Tomalia, D. A., et al., (1990) Angew. Chem. Int. Ed. Engl. 29: 5305). Synthesis and utilization of dendrimers has been postulated in the art, and dendrimers have been utilized for chemotherapeutic drug targeting in vitro (see, e.g., P. Singh, et al., (1994) Clin. Chem. 40: 1845). The calumenin-specific targeting component should bind to calumenin or a portion of calumenin so as to decrease the activity of the enzyme in the targeted cancer cell. The calumenin-specific targeting component can be a nucleic acid that hybridizes specifically to sequences encoding calumenin or a portion of the calumenin polypeptide. Moreover, the calumenin-specific targeting component is selected from the group consisting of peptides, peptidomimetic compounds, small molecules specifically designed to bind calumenin, and inhibitors of calumenin. The aforementioned compounds are not intended to limit the range of compounds that can serve as the calumenin-specific targeting component, but are merely illustrative examples.

The calumenin-targeting agent can be an interfering RNA (RNAi) that specifically hybridizes to a segment or region of the calumenin nucleic acids expressed in the cancer cells. Ribonucleic acids used in RNAi to hybridize to target sequences can be of lengths between 10 to 20 bases, between 9 to 21 bases, between 7 to 23 bases, between 5 to 25 bases, between 25 to 35 bases, between 27 to 33 bases, and between 35 to 40 bases.

Following or at the time of treatment of a patient with calumenin-targeted therapy, chemotherapeutic treatment is administered. Non-limiting examples of useful chemotherapeutic drugs for treating a patient include Actinomycin, Adriamycin, Bleomycin, Busulfan, Capecitabine, Carboplatin, Carmustine, Cisplatin, Cladribine, Cyclophosphamide, Cytarabine, Dacarbazine, Dactinomycin, Daunorubicin, Docetaxel, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristine, and Vinorelbine. These drugs are commercially obtainable, e.g., from ScienceLab.com, Inc. (Kingwood, Tex.). Physician administered treatment with these chemotherapeutic drugs is well known in the art (see, e.g., Capers et al., (1993) Hosp. Pharm. 28(3):206-10).

Aspects of the invention additionally provide kits for detecting chemotherapeutic drug resistance in neoplastic cells. The kits include probes for the detection of calumenin and probes for the detection of multidrug resistance protein 1, BRCP, p53, vimentin, α-enolase, nucleophosmin, and HSC70. During the course of patient chemotherapeutic treatment, monitoring of calumenin, and other MDR-associated markers described herein, provides valuable information regarding the efficacy of the treatment and for avoiding the development of chemotherapeutic drug resistance. The kit can comprise a labeled compound or agent capable of detecting calumenin protein in a biological sample; as well as means for determining the amount of calumenin in the sample; and means for comparing the amount of calumenin in the sample with a standard (e.g., normal non-neoplastic cells or non-MDR neoplastic cells). The compound or agent can be packaged in a suitable container. The kit can further comprise instructions for using the kit to detect calumenin protein, as well as other MDR-associated markers. Such a kit can comprise, e.g., one or more antibodies that bind specifically to at least a portion of a calumenin protein on a neoplastic cell.

The kit can also contain nucleic acids that are capable of detecting calumenin expression in a cell sample. Non-limiting examples of nucleic acids include single-stranded RNA, double-stranded RNA, double-stranded DNA, single-stranded DNA, and RNA-DNA hybrids. Furthermore, nucleic acids can be labeled as described herein.

The kit contains a second probe for detection of MDR protein expression, which indicates the presence of chemotherapeutic drug resistance. These probes advantageously allow health professionals to obtain an additional data point to determine whether chemotherapeutic drug resistance exists. The probes can be labeled antibodies or fragments thereof capable of binding at least a portion of the chemotherapeutic drug resistance markers. Additionally, the probes can be nucleic acids capable of hybridizing to a region of a chemotherapeutic drug resistance marker. Multidrug resistance protein 1, BRCP, p53, vimentin, α-enolase, nucleophosmin, and HSC70 can be used as MDR proteins. However, other MDR proteins are known in the art and can be used in the present aspect of the invention (see, e.g., Ojima et al. (2005) J. Med. Chem. 48(6):2218-28; Matsumoto et al. (2005) J. Med. Invest. 52(1-2):41-8).

1.2 Targeting Agents

The present invention utilizes a calumenin-targeting agent for use in increasing the sensitivity of neoplasms to allow for improved efficacy of chemotherapeutic treatment. The present invention also utilizes calumenin-targeting agents for use in preventing or treating chemotherapeutic drug-resistant neoplasms. In some instances, targeting agents can be in the form of proteins (hereinafter termed “protein-targeting agents”). As used herein, the term “protein-targeting agents” means a protein molecule or fragment thereof that can interact, bind, or associate with a molecule in a sample. A protein-targeting agent can be a protein or polypeptide capable of binding a biological macromolecule such as a protein, nucleic acid, simple carbohydrate, complex carbohydrate, fatty acid, lipoprotein, and/or triacylglyceride. Exemplary protein targeting agents include natural ligands of a receptor, hormones, antibodies, and portions thereof. The techniques associated with the binding of ligands and hormones to proteins as targeting agents have been demonstrated previously (see, e.g., Cutting et al., (2004) J. Biomol. NMR. 30(2):205-10).

The invention provides protein-targeting agents that are composed of antibodies or fragments of antibodies that specifically bind to calumenin. The invention allows for antibodies to be immobilized on a solid support such as an antibody array where the support can be a bead or flat surface similar to a slide. An antibody microarray can determine the MDR protein expression of a chemotherapeutic drug-resistant cancer cell sample and the MDR protein expression of a multi-drug-sensitive control cell of the same tissue type. Alternatively, antibodies can be free in solution. Antibodies can also be conjugated to a non-limiting material such as magnetic compounds, paramagnetic compounds, proteins, nucleic acids, antibody fragments, or combinations thereof. In some embodiments, antibodies are used to inhibit calumenin to decrease the activity of the enzyme in a targeted cell, thereby increasing the chemosensitivity of the cell to chemotherapeutic treatments (see Lopez-Alemany et al. (2003) Am. J. Hematol. 72(4): 234-42).

Protein targeting agents, including antibodies, can be detectably labeled. As used herein, “detectably labeled” means that a targeting agent is operably linked to a moiety that is detectable. By “operably linked” is meant that the moiety is attached to the targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

Useful labels can be, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that can be colored, chemoluminescent, or magnetic. The signal is detectable by any suitable means, including spectroscopic, photochemical, biochemical, immunochemical, electrical, optical or chemical means. In certain cases, the signal is detectable by two or more means.

Labeled protein targeting agents allow detection of the level of expression of calumenin in a cancer cell sample. For example, protein-targeting agents can be labeled for detection using chemiluminescent tags affixed to amino acid side chains. Useful tags include, but are not limited to, biotin, fluorescent dyes such as Cy5 and Cy3, and radiolabels (see, e.g., Barry and Soloviev (2000) Proteomics. 4(12): 3717-3726). Tags can be affixed to the amino terminal portion of a protein or the carboxyl terminal portion of a protein (see, e.g., Mattison and Kenney, (2002) J. Biol. Chem., 277(13): 11143-11148; Berne et al., (1990) J. Biol. Chem. 265(32): 19551-9). Indirect detection means can also be used to identify the cell markers. Exemplary but non-limiting means include detection of a primary antibody using a fluorescently labeled secondary antibody, or a secondary antibody tagged with biotin such that it can be detected with fluorescently labeled streptavidin.

As used herein, a “nucleic acid targeting agent” is defined as a nucleic acid capable of binding to a target nucleic acid of complementary sequence through one or more types of chemical bonds, usually through complementary base pairing, usually through hydrogen bond formation. “Nucleic acid” refers to a polymer comprising 2 or more nucleotides and includes single-, double-, and triple-stranded polymers. “Nucleotide” refers to both naturally occurring and non-naturally occurring compounds and comprises a heterocyclic base, a sugar, and a linking group, such as a phosphate ester. For example, structural groups are added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid can be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

Moreover, a nucleic acid targeting agent can include natural (i.e., A, G, U, C, or T) or modified (7-deazaguanosine, inosine, etc.) bases. In addition, the bases in targeting agents can be joined by a linkage other than a phosphodiester bond, so long as it does not interfere with hybridization. Thus, nucleic acid targeting agents can be peptide nucleic acids in which the constituent bases are joined by peptide bonds rather than phosphodiester linkages. The nucleic acid targeting agents can be prepared by converting the RNA to cDNA using known methods (see, e.g., Ausubel et. al., Current Protocols in Molecular Biology, Wiley 1999). The targeting agents can also be cRNA (see, e.g., Park et. al., (2004) Biochem. Biophys. Res. Commun. 325(4): 1346-52).

Nucleic acid targeting agents can be produced from synthetic methods such as phosphoramidite methods, H-phosphonate methodology, and phosphite triester methods. Nucleic acid targeting agents can also be produced by PCR methods. Such methods produce cDNA and cRNA sequences complementary to the mRNA. Such nucleic acid targeting agents can be detectably labeled, with, e.g., fluorophores (e.g., fluorescein (FITC), phycoerythrin, rhodamine), chemical dyes, or compounds that are radioactive, chemoluminescent, magnetic, paramagnetic, promagnetic, or enzymes that yield a product that can be colored, chemiluminescent, or magnetic. The signal is detectable by any suitable means, including spectroscopic, photochemical, biochemical, immunochemical, electrical, optical or chemical means. In certain cases, the signal is detectable by two or more means. In certain embodiments, nucleic acid labels include fluorescent dyes, radiolabels, and chemiluminescent labels, which are examples that are not intended to limit the scope of the invention (see, e.g., Yu, et al., (1994) Nucleic Acids Res. 22(16): 3226-3232; Zhu, et al., (1994) Nucleic Acids Res. 22(16): 3418-3422).

Nucleic acid targeting agents can be detectably labeled using fluorescent labels. Non-limiting examples of fluorescent labels include 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein). These labels can be commercially obtained, e.g., from PerkinElmer Corp. (Boston, Mass.).

Other useful dyes are chemiluminescent dyes and can include, without limitation, biotin conjugated DNA nucleotides and biotin conjugated RNA nucleotides. Labeling of nucleic acid targeting agents can be accomplished by any means known in the art. (see, e.g., CyScribe™ First Strand cDNA Labeling Kit (#RPN6200, Amersham Biosciences, Piscataway, N.J.). The label can be added to the target nucleic acid(s) prior to, or after the hybridization. So called “direct labels” are detectable labels that are directly attached to, or incorporated into, the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid can be biotinylated before the hybridization. After hybridization, an avidin-conjugated fluorophore binds the biotin bearing hybrid duplexes providing a label that is easily detected. (see, e.g., Laboratory Techniques in Biochemistry and Molecular Biology, Vol. 24: Hybridization With Nucleic Acid Targeting agents, P. Tijssen, ed. Elsevier, N.Y., (1993)).

Nucleic acid targeting agents can also be immobilized on a solid support such as glass, polystyrene, nylon, and PVDF membrane. In these embodiments, the nucleic acid targeting agent is contacted by an isolated cell sample, and subsequently allowed to hybridize to the target nucleic acid in the sample. In certain embodiments, a microarray is utilized to detect calumenin expression levels. Microarray technology has been utilized to determine the expression levels of various other genes, and the techniques are well known in the art (see, e.g., Zhang et al. (2004) Proc. Nat. Acad. Sci. USA. 101(39): 14168-14173).

Alternatively, expression levels for the calumenin mRNA can be determined using techniques known in the art, such as, but not limited to, quantitative RT-PCR and RNA blotting (see, e.g., Rehman et al. (2004) Hum. Pathol. 35(11): 1385-91; Yang et al. (2004) Mol. Biol. Rep. 31(4): 241-8). Such examples are not intended to limit the potential means for determining the expression of a gene marker in a breast cancer cell sample.

In addition, aptamers can be calumenin-targeting agents. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target of the present invention is calumenin, and hence the term calumenin aptamer or nucleic acid ligand is used. Inhibition of the target by the aptamer can occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers can be comprised of multiple ribonucleotide units, deoxyribonucleotide units, or a mixture of both types of nucleotide residues. Aptamers can further comprise one or more modified bases, sugars or phosphate backbone units as described above.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. One method is the 2′-O-tert-butyldimethylsilyl (TBDMS) protection strategy for the ribonucleotides (Usman et al., (1987) J. Am. Chem. Soc., 109: 7845-7854), and all the required 3′-O-phosphoramidites are commercially available. In addition, aminomethylpolystyrene can be used as the support material due to its advantageous properties (McCollum and Andrus (1991) Tetrahedron Lett. 32:4069-4072). Fluorescein can be added to the 5′-end of a substrate RNA during the synthesis by using commercially available fluorescein phosphoramidites.

In general, an aptamer oligomer can be synthesized using a standard RNA cycle. Upon completion of the assembly, all base labile protecting groups are removed by an eight-hour treatment at 55° C. with concentrated aqueous ammonia/ethanol (3:1 v/v) in a sealed vial. The ethanol suppresses premature removal of the 2′-O-TBDMS groups that would otherwise lead to appreciable strand cleavage at the resulting ribonucleotide positions under the basic conditions of the deprotection (Usman et al., (1987) J. Am. Chem. Soc., 109: 7845-7854). After lyophilization, the TBDMS protected oligomer is treated with a mixture of triethylamine trihydrofluoride/triethylamine/N-methylpyrrolidinone for 2 hours at 60° C. to afford fast and efficient removal of the silyl protecting groups under neutral conditions (see Wincott et al., (1995) Nucleic Acids Res., 23:2677-2684). The fully deprotected oligomer can then be precipitated with butanol according to the procedure of Cathala and Brunel ((1990) Nucleic Acids Res., 18:201). Purification can be performed either by denaturing polyacrylamide gel electrophoresis or by a combination of ion exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. Traces of residual salts can then be removed using small disposable gel filtration columns that are commercially available. As a final step the authenticity of the isolated oligomers can be checked by matrix assisted laser desorption mass spectrometry (Pieles et al., (1993) Nucleic Acids Res., 21:3191-3196) and by nucleoside base composition analysis.

The disclosed aptamers can also be produced through enzymatic methods, when the nucleotide subunits are available for enzymatic manipulation. For example, the RNA molecules can be made through in vitro RNA polymerase T7 reactions. They can also be made by strains of bacteria or cell lines expressing T7, and then subsequently isolated from these cells. As discussed below, the disclosed aptamers can also be expressed in cells directly using vectors and promoters.

The aptamers, like other nucleic acid molecules of the invention, can further contain chemically modified nucleotides. One issue to be addressed in the diagnostic or therapeutic use of nucleic acids is the potential rapid degradation of oligonucleotides in their phosphodiester form in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand (see, e.g., U.S. Pat. No. 5,660,985).

The stability of the aptamer can be greatly increased by the introduction of such modifications and as well as by modifications and substitutions along the phosphate backbone of the RNA. In addition, a variety of modifications can be made on the nucleobases themselves, which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers can also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer can further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer can still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-aminoadenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Examples of other modified base variants known in the art include, without limitation, e.g., 4-acetylcytidine, 5-(carboxyhydroxylmethyl) uridine, 2′-methoxycytidine, 5-carboxymethylaminomethyl-2-thioridine, 5-carboxymethylaminomethyluridine, dihydrouridine, 2′-O-methylpseudouridine, b-D-galactosylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methylcarbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, and wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941. Examples of modified nucleoside and nucleotide sugar backbone variants known in the art include, without limitation, those having, e.g., 2′ ribosyl substituents such as F, SH, SCH3, OCN, Cl, Br, CN, CF3, OCF3, SOCH3, SO2, CH3, ONO2, NO2, N3, NH2, OCH2CH2OCH3, O(CH2)2ON(CH3)2, OCH2OCH2N(CH3)2, O(C1-10 alkyl), O(C2-10 alkenyl), O(C2-10 alkynyl), S(C1-10 alkyl), S(C2-10 alkenyl), S(C2-10 alkynyl), NH(C1-10 alkyl), NH(C2-10 alkenyl), NH(C2-10 alkynyl), and O-alkyl-O-alkyl. Desirable 2′ ribosyl substituents include 2′-methoxy (2′-OCH3), 2′-aminopropoxy (2′ OCH2CH2CH2NH2), 2′-allyl (2′-CH2-CH═CH2), 2′-O-allyl (2′-O—CH2-CH═CH2), 2′-amino (2′-NH2), and 2′-fluoro (2′-F). The 2′-substituent can be in the arabino (up) position or ribo (down) position.

Aptamers can be made up of nucleotides and/or nucleotide analogs such as described above, or a combination of both, or are oligonucleotide analogs. Aptamers can contain nucleotide analogs at positions, which do not affect the function of the oligomer to bind calumenin.

There are several techniques that can be adapted for refinement or strengthening of the nucleic acid ligands binding to a particular target molecule or the selection of additional aptamers. One technique, generally referred to as “in vitro genetics” (see Szostak (1992) TIBS, 19:89), involves isolation of aptamer antagonists by selection from a pool of random sequences. The pool of nucleic acid molecules from which the disclosed aptamers can be isolated can include invariant sequences flanking a variable sequence of approximately twenty to forty nucleotides. This method has been termed Selective Evolution of Ligands by Exponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

Other modifications useful for producing aptamers of the invention are known to one of ordinary skill in the art. Such modifications can be made post-SELEX process (modification of previously identified unmodified ligands) or by incorporation into the SELEX process. It has been observed that aptamers, or nucleic acid ligands, in general, are most stable, and therefore efficacious when 5′-capped and 3′-capped in a manner which decreases susceptibility to exonucleases and increases overall stability.

Calumenin-targeting agents are specifically targeted to a neoplasm to prevent detection of calumenin activity in normal cells of the patient or in the serum of the patient. Likewise, calumenin-targeting agents are targeted to specifically decrease the level of expression of calumenin in neoplastic cells. Targeting mechanisms include non-limiting techniques such as conjugating the calumenin-targeting agent to an agent that binds to a cancer cell marker (hereinafter termed “cancer cell targeting components”). Cancer cell targeting components include, but are not limited to, antibodies or binding fragments thereof, nucleic acids, peptides, small molecules, and peptidomimetic compounds. Cancer cell targeting components can be conjugated directly to the calumenin-targeting agent, for example, through covalent bonding to, e.g., carboxyl, phosphoryl, sulfhydryl, carbonyl, and hydroxyl groups using chemical techniques known in the art. Alternatively, cancer cell targeting components and calumenin-targeting agents can be conjugated to functionalized chemical groups on non-limiting examples of inert supports such as polyethylene glycol, glass, synthetic polymers such as polyacrylamide, polystyrene, polypropylene, polyethylene, or natural polymers such as cellulose, Sepharose, or agarose, or conjugates with enzymes. Chemical conjugation techniques are well known in the art. Non-limiting examples of cancer cell markers that can be used for targeting of calumenin-targeting agent include Pgp-1, MRP1, BIP, BRCP, HSC70, nucleophosmin, vimentin, and HSP90.

Alternatively, the calumenin-targeting agent can be targeted to a neoplasm through variety of invasive procedures. In the context of some aspects of the present invention, such procedures include catheterization through an artery of a patient and depositing the calumenin-targeting agent within the tumor site. A surgeon can also apply the calumenin-targeting agent to the neoplasm by making an incision into the patient at a site that allows access to the tumor for placement of the calumenin-targeting agent into, onto, or in close proximity to, the tumor. In some instances, a subject can also be intubated with subsequent introduction of the calumenin-targeting agent into the tumor site through the tube. In other embodiments, the calumenin-targeting agent can be administered to a patient orally, subcutaneously, intramuscularly, intravenously, or interperitoneally.

The calumenin-targeting agent can be incorporated into a liposome before it is used. The term “liposome,” as used herein, refers to an artificial phospholipid bilayer vesicle. The liposome formulation can be used to facilitate lipid bilayer fusion with a target cell, thereby allowing the contents of the liposome or proteins associated with its surface to be brought into contact with the neoplastic cell. Liposomes can have antibodies associated with their bilayers that allow binding to targets on the neoplastic cell surface (hereinafter termed “immunoliposome”). Non-limiting examples of neoplastic cell targets to which such antibodies are specifically directed include Pgp-1, MRP1, BIP, BRCP, HSC70, nucleophosmin, vimentin, and HSP90. Antibodies for these cell markers can be obtained commercially (e.g., Research Diagnostics, Inc., Flanders, N.J.; and Abcam, Inc., Cambridge, Mass.).

1.3 Antibodies for Detection of Calumenin

Aspects of the present invention utilize antibodies directed against calumenin for use in diagnosis, detection, and prevention of chemotherapeutic drug-resistant cancer cells. Antibodies can also be used in the treatment of neoplasms or neoplastic cells by decreasing the activity of calumenin in a neoplasm. Calumenin antibodies can be administered to a patient orally, subcutaneously, intramuscularly, intravenously, or interperitoneally.

The invention also utilizes polyclonal antibodies for the detection of calumenin. As used herein, the term “polyclonal antibodies” means a population of antibodies that can bind to multiple epitopes on an antigenic molecule. A polyclonal antibody is specific to a particular epitope on an antigen, while the entire pool of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology, Vol. 1, pp. 4.2.1-4.2.9, John Wiley & Sons, Inc., 1996).

The term “monoclonal antibody,” as used herein, refers to an antibody obtained from a population of substantially homogenous antibodies, i.e., the individual antibodies comprising the population are identical except for possible naturally occurring mutations that can be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention.

The monoclonal antibodies herein include hybrid and recombinant antibodies produced by splicing a variable (including hypervariable) domain of an anti-calumenin antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al. (1990) Nature 348:552-554).

Alternative methods for producing antibodies can be used to obtain high affinity antibodies. Antibodies for calumenin can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Cannard and Georgiou (2000) Ann. Rev. Biomed. Eng. 2: 339-76; U.S. Pat. Nos. 4,833,077; 5,811,524; 5,958,765; 6,413,771; and 6,537,809).

Antibodies are used to bind to calumenin to decrease the activity of calumenin in cancer cells. In some aspects of the invention, the antibody binds to calumenin in domains vital to its activity. For instance, monoclonal or polyclonal antibodies directed to its calcium-binding domain can decrease the activity of calumenin sufficiently to produce a desired inhibitory effect. Also, antibodies can be used to decrease the interaction of calumenin with various proteins in the endoplasmic reticulum or Golgi complex. Techniques for inhibiting protein activity using antibodies are generally known in the art, and have been utilized to inhibit proteins such as PLTP, CETP, and other cell surface and intracellular proteins (see, e.g., Saito et al. (1999) J. Lipid Res. 40: 2013-2021; Cui et al. (2003) Eur. J. Biochem. 270: 3368-3376; Siggins et al. (2003) J. Lipid Res. 44: 1698-1704; Du et al. (1996) J. Biol. Chem. 271(13): 7362-7367).

1.4 RNA Interference

Aspects of the invention further allow for the treatment of a patient with a neoplasm, which includes chemotherapeutic drug-resistant neoplasms, by increasing the sensitivity of the neoplasm to a chemotherapeutic drug using RNA interference (“RNAi”). As used herein, the term “RNA interference” refers to the blocking or preventing of cellular production of a particular protein by stopping the mechanisms of translation using small RNAs that hybridize to complementary sequences in a target mRNA. RNAi is essentially a type of anti-sense strategy for preventing RNA translation, even though the technology has slightly different mechanisms of action than general anti-sense strategies. Anti-sense RNA strategies utilize the single-stranded nature of mRNA in a cell to block or interfere with translation of the mRNA into a protein. Anti-sense technology has been the most commonly described approach in protocols to achieve gene-specific interference. For anti-sense strategies, stoichiometric amounts of single-stranded nucleic acid complementary to the messenger RNA for the gene of interest are introduced into the cell.

The RNA can comprise one or more strands of polymerized ribonucleotide. It can include modifications to either the phosphate-sugar backbone or the nucleoside. For example, the phosphodiester linkages of natural RNA can be modified to include at least one of a nitrogen or sulfur heteroatom. For example, structural groups can be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid can be substituted or modified, for example with methyl phosphonates or O-methyl phosphates. Bases and sugars can also be modified, as is known in the art. RNA can also be modified to include “peptide nucleic acids” in which native or modified nucleic acid bases are attached to a polyamide backbone. Modifications in RNA structure can be tailored to allow specific genetic inhibition while avoiding a general panic response in some organisms, which is generated by dsRNA. Likewise, bases can be modified to block the activity of adenosine deaminase. RNA can be produced enzymatically or by partial/total organic synthesis, any modified ribonucleotide can be introduced by in vitro enzymatic or organic synthesis.

Methods of using siRNA to inhibit gene expression are well known in the art (see e.g., U.S. Pat. No. 6,506,559). Typically, complementary RNA sequences that can hybridize to a specific region of the target RNA are introduced into the cell. RNA annealing to the target transcripts allows the internal machinery of the cell to cut the dsRNA sequences into short segments. It is this machinery that allows sub-stoichiometric numbers of siRNA molecules to be used to silence a particular gene. Such mechanisms have been utilized in in vitro and in vivo studies of human genes (see, e.g., Mizutani et al. (2002) J. Biol. Chem. 277(18): 15859-64; Wang et al. (2005) Breast Cancer Res. 7(2): R220-8). In particular, the c-myc gene was inhibited in MCF7 breast cancer cell lines using the RNA interference technique (see Wang et al. (2005) Breast Cancer Res. 7(2): R220-8).

Interfering RNAs can be obtained by any means known in the art. For example, they can be synthetically produced using the Expedite™ Nucleic Acid Synthesizer (Applied Biosystems, Foster City, Calif.) or other similar devices (see, e.g., Applied Biosystems, Foster City, Calif.). Synthetic oligonucleotides also can be produced using methods well known in the art such as phosphoramidite methods (see, e.g., Pan et. al., (2004) Biol. Proc. Online. 6:257-262), H-phosphonate methodology (see, e.g., Agrawal et. al., (1987) Tetrahedron Lett. 28(31): 3539-3542) and phosphite triester methods (Finnan et al. (1980) Nucleic Acids Symp. Ser. (7): 133-45).

1.5 Diagnostic Methods for Detection of Calumenin

Aspects of the invention allow the identification of patients having MDR neoplastic cells. For example, where the patient identified as having such cells is a patient in remission of cancer or is being treated for cancer (e.g., a patient suffering from breast cancer, ovarian cancer, prostate cancer, leukemia, etc.), the invention allows identification of these patients prior to resurgence and/or progression of their cancer, as well as allows the monitoring of these patients during treatment with a drug, such that the treatment regimen can be altered.

The diagnostic applications of the invention include probes and other detectable agents that are joined to a calumenin-targeting agent, such as an anti-calumenin antibody. Conjugation of such agents to the targeting agent can be accomplished by, e.g., covalent bonding to non-limiting active groups such as carbonyls, carboxyls, amines, amides, hydroxyls, and sulfhydryls. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y. 1999).

In accordance with the invention, a detectably labeled targeting agent of the invention includes a targeting agent that is conjugated to a detectable moiety. Another detectably labeled targeting agent of the invention is a fusion protein, where one component is the targeting agent and the other component is a detectable label. Yet another non-limiting example of a detectably labeled targeting agent is a first fusion protein comprising a targeting agent and a first moiety with high affinity to a second moiety, and a second fusion protein comprising a second moiety and a detectable label. For example, a targeting agent that specifically binds to a calumenin protein can be operably linked to a streptavidin moiety. A second fusion protein comprising a biotin moiety operably linked to a fluorescein moiety can be added to the targeting agent-streptavidin fusion protein, where the combination of the second fusion protein to the targeting agent-streptavidin fusion protein results in a detectably labeled targeting agent (i.e., a targeting agent operably linked to a detectable label). Detectable labels have been described above.

Useful detectable targeting agents are labeled antibodies, and derivatives and analogs thereof, which specifically bind to calumenin polypeptide (see Section 1.3). These antibodies can be used for diagnostic purposes to detect, diagnose, or monitor diseases and/or disorders associated with the aberrant expression of calumenin. The invention provides for the detection of aberrant expression of calumenin (a) assaying the expression of the polypeptide of interest in cells or cell surface membrane fractions of an individual using one or more antibodies specific to calumenin and (b) comparing the level of gene expression with a standard gene expression level, whereby an increase or decrease in the assayed calumenin expression level compared to the standard expression level is indicative of aberrant expression. For example, where chemotherapeutic drug resistance in a neoplastic cell is to be detected, the standard expression level to which comparison should be made is a neoplastic cell of the same or similar origin or cell type, which has not previously demonstrated characteristics associated with chemotherapeutic drug resistance. Similarly, where neoplasia in a test cell is to be detected, the standard expression level to which comparison should be made is a non-neoplastic cell of the same or similar origin or cell type.

The presence of increased calumenin expression in biopsied tissue or test cell from an individual can indicate a predisposition for the development of chemotherapeutic drug resistance, or can provide a means for detecting chemotherapeutic drug resistance prior to the appearance of actual clinical symptoms. A more definitive diagnosis of this type allows health professionals to employ preventative measures or aggressive treatment earlier, thereby preventing the development or further progression of the cancer. Information of this type allows for clinicians to tailor their treatment choices accordingly, potentially preventing development of neoplastic disease in additional tissues within the patient.

Antibodies directed to calumenin are also useful to assay protein levels in a biological sample using classical immunohistological methods known to those of skill in the art (see, e.g., Jalkanen et al., (1985) J. Cell. Biol. 101:976-985; Jalkanen et al. (1987) J. Cell. Biol. 105:3087-3096). Other antibody-based methods useful for detecting calumenin protein expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹¹²In), and technetium (⁹⁹Tc); luminescent labels, such as luminol; and fluorescent labels, such as fluorescein and rhodamine, and biotin.

1.6 Liposome

Another strategy that can be employed for delivery of calumenin-targeting agent is the use of immunoliposomes. Immunoliposomes incorporate antibodies against tumor-associated antigens into liposomes, which carry the therapeutic agent, such as the calumenin-targeting agent, or an enzyme that activates an otherwise inactive prodrug (see, e.g., Lasic et al. (1995) Science 267: 1275-76). Immunoliposomal drugs can be used to successfully target and enhance anti-cancer efficacy (see, e.g., Maruyama et al. (1990) J. Pharm. Sci. 74: 978-84); Maruyama et al. (1995) Biochim. Biophys. Acta 1234: 74-80; Otsubo et al. (1998) Antimicrob. Agents Chemother. 42: 40-44; Lopes de Menezes et al. (1998) Cancer Res. 58: 3320-30).

Calumenin targeting agents can be incorporated into the membrane of the liposome through mechanisms known in the art (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). In addition, calumenin-targeting agents can be associated with the outside of a liposome through covalent linkages to PEG polymers (see, e.g., Medina et al. (2004) Curr. Pharm. Des. 10(24): 2981-9). Furthermore, targeting agents can be incorporated into the hydrated inner compartment of the liposome (see, e.g., Medina et al. (2004) Curr. Pharm. Des. 10(24): 2981-9). A combination of the above mentioned liposome delivery methods can be used in a therapeutic composition.

Alternatively, modified LDL can be used as tumor-specific ligands in targeting liposomal formulations containing calumenin-targeting agents. For example, folate-coupled liposomes can be used to target therapeutics to tumors, which overexpress the folate receptor (Lee and Low (1994) J. Biol. Chem. 269: 3198-204; Lee and Low (1995) Biochim. Biophys. Acta 1233: 134-44; Rui et al. (1998) J. Am. Chem. Soc. 120: 11213-18; Gabizon et al. (1999) Bioconj. Chem. 10: 289-98). Transferrin has been employed as a targeting ligand to direct liposomal drugs to various types of cancer cell in vivo (Ishida and Maruyama (1998) Nippon Rinsho 56: 657-62; Kirpotin et al. (1997) Biochem. 36: 66-75). PEG-immunoliposomes with anti-transferring antibodies coupled to the distal ends of the PEG associate with C6 glioma cells in vitro and significantly increased gliomal doxorubicin uptake after treatment with the tumor-specific long-circulating liposomes containing doxorubicin (Eavarone et al. (2000) J. Biomed. Mater. Res. 51: 10-14).

Methods of delivering chemotherapeutic drugs and siRNA in vivo are known in the art (see, e.g., Mewani et al. (2004) Int. J. Oncol. 24(5): 1181-8; Chien et al. (2005) Cancer Gene Ther. 12(3): 3221-8). Liposomes have also been used for the targeted delivery of chemotherapeutic drugs, toxins, and labels (see, e.g., Pakunlu et al. (2004) Cancer Res. 64(17): 6214-24; Shimizu et al. (2002) Biol. Pharm. Bull. 25(6): 783-6; Zheng and Tan (2004) World J. Gastroenterol. 10(17): 2563-6). Liposome formulations for the delivery of chemotherapeutics and siRNA can be obtained from commercial suppliers, e.g., Eurogentec, Ltd. (Southampton, Hampshire, UK). In addition, methods for producing liposome micelle/chemotherapeutic formulations are well known in the art. For example, therapeutic drug micelles can be formed by combining a therapeutic drug and a phosphatidyl glycerol lipid derivative (PGL derivative). Briefly, the therapeutic drug and PGL derivative are mixed in a range of 1:1 to 1:2.1 to form a therapeutic drug mixture. Alternatively, the range of therapeutic drug to PGL derivative is 1:1.2; or 1:1.4; or 1:1.5; or 1:1.6; or 1:1.8 or 1:1.9 or 1:2.0 or 1:2.1. The mixture is then combined with an effective amount of at least a 20% organic solvent such as an ethanol solution to form micelles containing the therapeutic drug. Methods for inclusion of an antibody or tumor targeting ligand into the micelle formulation to produce immunoliposomes are known in the art and described further below. For example, methods for preparation and use of immunoliposomes are described in U.S. Pat. Nos. 4,957,735, 5,248,590, 5,464,630, 5,527,528, 5,620,689, 5,618,916, 5,977,861, 6,004,534, 6,027,726, 6,056,973, 6,060,082, 6,316,024, 6,379,699, 6,387,397, 6,511,676 and 6,593,308.

As used herein, the term “phosphatidyl glycerol lipid derivative (PGL derivative)” is any lipid derivative having the ability to form micelles and have a net negatively charged head group. This includes but is not limited to dipalmitoyl phosphatidyl glycerol (DPPG), dimyristoyl phosphatidyl glycerol, and dicapryl phosphatidyl glycerol. In one aspect, phosphatidyl derivatives with a carbon chain of 10 to 28 carbons and having unsaturated side aliphatic side chain are within the scope of this invention. The complexing of a therapeutic drug with negatively-charged phosphatidyl glycerol lipids having variations in the molar ratio giving the particles a net positive (1:1) neutral (1:2) or slightly negative (1:2.1) charge will allow targeting of different tissues in the body after administration. However, complexing of a therapeutic drug with negatively charged PGL has been shown to enhance the solubility of the therapeutic drug in many instances, thus reducing the volume of the drug required for effective antineoplastic therapy. In addition, the complexing of a therapeutic drug and negatively charged PGL proceeds to very high encapsulation efficiency, thereby minimizing drug loss during the manufacturing process. These complexes are stable, do not form precipitates and retain therapeutic efficacy after storage at 4° C. for at least four months. In order to achieve maximum therapeutic efficacy by avoiding rapid clearance from the blood circulation by the reticuloendothelial system (RES), immunoliposomal drug formulations incorporate components such as polyethylene glycol (PEG) (see, e.g., Klibanov et al. (1990) FEBS Lett. 268: 235-7; Mayuryama et al. (1992) Biochim. Biophys. Acta 1128: 44-49; Allen et al. (1991) Biochim. Biophys. Acta 1066: 29-36). Long-circulating immunoliposomes can be classified into two types: those with antibodies coupled to a lipid head growth (Maruyama et al. (1990) J. Pharm. Sci. 74: 978-84); and those with antibodies coupled to the distal end of PEG (Maruyama et al. (1997) Adv. Drug Del. Rev. 24: 235-42). In certain instances, it is advantageous to place the tumor-specific antibodies at the distal end of the PEG polymer to obtain efficient target binding by avoiding steric hindrance from the PEG chains.

1.7 Calumenin-Directed Cancer Therapies

The invention provides treatments that increase the sensitivity of cancer cells to chemotherapeutic drugs. Moreover, the invention provides for treatment or prevention of multi-drug-resistant cancer, including, but not limited to, neoplasms, tumors, or metastases, and particularly chemotherapeutic drug-resistant forms thereof by the administration of therapeutically or prophylactically effective amounts of anti-calumenin antibodies or nucleic acid molecules encoding said antibodies. In addition, calumenin therapies include nucleic acids complementary to a sequence encoding the calumenin protein. Calumenin therapies are utilized to decrease the activity of calumenin in a cancer cell, thereby improving the efficacy of the treatment regime, and, in some instances, changing the chemotherapeutic drug-resistant phenotype of the cancer.

Examples of types of cancer and proliferative disorders to be treated with the calumenin-targeted therapeutics of the invention include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma (but not including squamous cell carcinomas of the cervix or of cervical origin), basal cell carcinoma, adenocarcinoma, renal cell carcinoma, hepatoma, Wilms' tumor, cervical cancer excluding cervix squamous cell carcinoma, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to individuals with breast cancer (e.g., breast adenocarcinoma, breast carcinoma, ductal carcinoma in situ, ductal carcinoma, invasive ductal carcinoma, Paget's Disease of the Nipple, lobular carcinoma, lobular carcinoma in situ, invasive lobular carcinoma, inflammatory breast cancer, medullary carcinoma, tubular carcinoma, cribriform carcinoma, papillary carcinoma, phyllodes tumor). In another embodiment, therapeutic compounds of the invention can be administered to a subject suffering from ovarian cancer (e.g., serous carcinoma, ovarian adenocarcinoma, mucinous carcinoma, endometrioid carcinoma, clear cell carcinoma, Brenner carcinoma, mature cystic teratoma, monodermal teratoma, immature teratoma, dysgerminoma, embryonal carcinoma, granulosa cell carcinoma). The treatment and/or prevention of chemotherapeutic drug-resistant or cancer includes, but is not limited to, alleviating symptoms associated with cancer, the inhibition of the progression of cancer, the promotion of the regression of cancer, and the promotion of the immune response.

The calumenin therapeutics can be administered in combination with other types of cancer treatments (e.g., radiation therapy, chemotherapy, hormonal therapy, immunotherapy and anti-tumor agents). Examples of anti-tumor agents include, but are not limited to, ifosfamide, paclitaxel, taxanes, topoisomerase I inhibitors (e.g., CPf-11, topotecan, 9-AC, and GG-211), gemcitabine, vinorelbine, oxaliplatin, 5-fluorouracil (5-FU), leucovorin, vinorelbine, Actinomycin, Adriamycin, Altretamine, Asparaginase, Bleomycin, Busulfan, Capecitabine, Carboplatin, Carmustine, Cisplatin, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristine, Vinorelbine, and temodal. Calumenin-targeting agents can be administered to a patient for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of the anti-tumor agent to the subject.

Calumenin-targeted therapeutics described herein, can be administered to a subject, including mammals such as humans, for the prevention or treatment of chemotherapeutic drug resistance prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs described herein. Nucleic acids complementary to calumenin messenger RNA are administered to an animal, including mammals such as humans, prior to (e.g., 1 min., 15 min., 30 min., 45 min., 1 hour, 2 hours, 4 hours, 6 hours, 8 hours, 12 hours, 24 hours, 2 days, or 1 week before), subsequent to (e.g., 1 min., 15 min., 30 min., 45 min., 1 hour, 2 hours, 4 hours, 6 hours, 8 hours, 12 hours, 24 hours, 2 days, or 1 week after), or concomitantly with the administration of chemotherapeutic drugs. The nucleic acids can be incorporated into a liposome for transport into a cell.

1.9 Pharmaceutical Formulations and Methods of Treatment

The present invention provides for both prophylactic and therapeutic methods of treating a subject having a neoplasm by increasing the sensitivity of the neoplasm to the chemotherapeutic treatment chosen by the physician. In certain cases, the present invention provides methods, both therapeutic and prophylactic, of treating a subject that suffers from a chemotherapeutic drug-resistant neoplasm. For both non-resistant cancer and resistant cancer, administration of a prophylactic agent can occur prior to the manifestation of symptoms characteristic of the neoplasm, such that development of the neoplasm is prevented or, alternatively, delayed in its progression. In general, the prophylactic or therapeutic methods comprise administering to the subject an effective amount of a compound, which comprises a calumenin binding component that is capable of binding to calumenin present in neoplastic, and particularly chemotherapeutic drug-resistant neoplastic, cells and which compound is linked to a therapeutic component. The calumenin binding component or agent binds to the calumenin expressed in the neoplastic cells and prevents calumenin activity in the cells, thereby rendering the cells susceptible to a chemotherapeutic treatment.

Calumenin-binding components can be targeted to neoplastic cells using a variety of targeting means. In some instances, the targeting component can be an antibody that binds to a neoplastic cell marker. The calumenin binding component can be targeted to the neoplastic cells by vimentin, nucleophosmin or HSC70 antibodies, for example. Examples of calumenin-targeting components include monoclonal anti-vimentin antibodies and fragments thereof. In addition to targeting and binding components, formulations can include cell internalization components such as liposomes and dendrimers. Subsequent to calumenin internalization into a neoplastic cell, therapeutic components can be administered to a patient to kill the neoplastic cell. Examples of suitable therapeutic components include traditional chemotherapeutic agents such as Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristine, and Vinorelbine.

For such therapy, the compounds of the invention can be formulated for a variety of loads of administration, including systemic and topical or localized administration. Techniques and formulations generally can be found in Remmington's Pharmaceutical Sciences, Meade Publishing Co., Easton, Pa. For systemic administration, injection is used, including intramuscular, intravenous, intraperitoneal, and subcutaneous. For injection, the compounds of the invention can be formulated in liquid solutions, including in physiologically compatible buffers such as Hank's solution or Ringer's solution. In addition, the compounds can be formulated in solid form and redissolved or suspended immediately prior to use. Lyophilized forms are also included.

For oral administration, the pharmaceutical compositions can take the form of, for example, tablets or capsules prepared by conventional means with pharmaceutically acceptable excipients such as targeting agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents can be used to facilitate permeation. Transmucosal administration can be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a therapeutic and gene delivery system for the calumenin-targeted therapeutic can be introduced into a patient by any of a number of methods, each of which is familiar in the art. For instance, a pharmaceutical preparation of the calumenin-targeted therapeutic can be introduced systemically, e.g., by intravenous injection.

The pharmaceutical preparation of the calumenin-targeted therapeutic compound of the invention can consist essentially of the compound in an acceptable diluent, or can comprise a slow release matrix in which the gene delivery vehicle or compound is imbedded.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

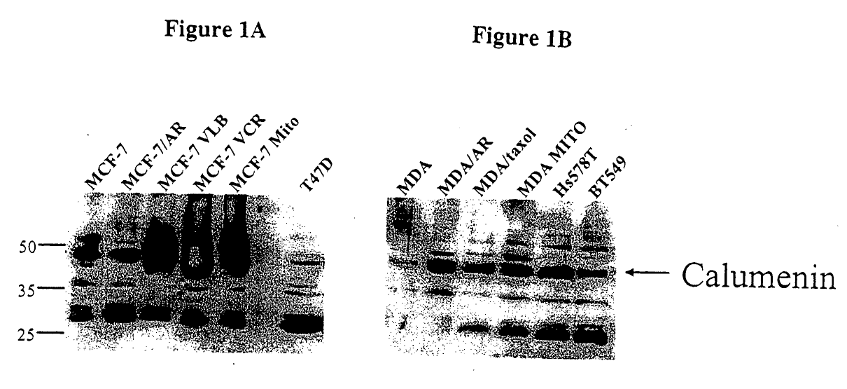

To demonstrate the methods according to the invention, a calumenin-targeting agent was prepared and tested for its ability to increase the sensitivity of various cancer cell samples to chemotherapeutic drugs. As a first step to elucidating the role that calumenin has in chemotherapeutic drug resistance, the levels of expression of calumenin were determined in resistant and non-resistant MCF-7 and MDA breast cancer cell lines. Cell extracts were prepared from resistant and nonresistant cell lines, and immunoblotted using anti-calumenin antibodies according to procedures described below. The non-resistant and resistant MCF-7 and MDA cells showed expression of a doublet at approximately 50 kD, which corresponds to previous reports concerning calumenin protein expression (FIGS. 1A and 1B, see Yabe et al. (1997) J. Biol. Chem. 272(29): 18232-18239). Of particular interest, MCF-7 cell lines resistant to vincristine, vinblastine, and mitoxantrone had higher calumenin expression levels than non-resistant MCF-7 cell lines (FIG. 1A). The increased levels of expression for calumenin in MCF-7 resistant cell lines suggested that calumenin could be a cell marker for chemotherapeutic drug resistance in breast cancer cells lines.

In addition to MCF-7 cell lines, the breast adenocarcinoma cell line MDA showed differential calumenin expression that was dependent on whether the cells had developed resistance to a particular chemotherapeutic drug. MDA cell lines resistant to adriamycin, taxol, and mitoxantrone had significantly increased levels of expression of calumenin protein as compared to non-resistant MDA cell lines (FIG. 1B). These results confirmed the results of the MCF-7 experiments, indicating that calumenin is a marker for chemotherapeutic drug resistance in certain cancer types.

To determine the potential for utilizing calumenin silencing in treating or improving the efficacy of certain chemotherapeutic treatments, several short nucleotide sequences were used to silence RNA expression. Two sequences were designed that corresponded to a region that is highly specific for the calumenin mRNA (Table 1). Two additional chemically modified Stealth siRNA duplexes were designed using RNAi designer resources (Invitrogen Corp., Carlsbad, Calif.). All sequences are shown in Table 1.

TABLE 1

Small Interfering RNA Duplexes
Targeting Calumenin

| | | SEQ. |
|---|---|---|
| | | ID |
| siRNA Duplex | Sequence | NO: |
| Calumenin | 5′-GAAGGACCGUGUACAUCAU-3′ | 1 |
| siRNA | | |
| (Sense) | | |
| Calumenin | 5′-AUGAUGUACACGGUCCUUC-3′ | 2 |
| siRNA | | |
| (Anti- | | |
| sense) | | |
| Calu-1 | 5′-GGGUGCUGAAGAAGCAAAGACCUUU-3′ | 3 |
| Stealth | | |
| siRNA | | |
| (Sense) | | |
| Calu-1 | 5′-AAAGGUCUUUGCUUCUUCAGCACCC-3′ | 4 |
| Stealth | | |
| siRNA | | |
| (Anti- | | |
| sense) | | |

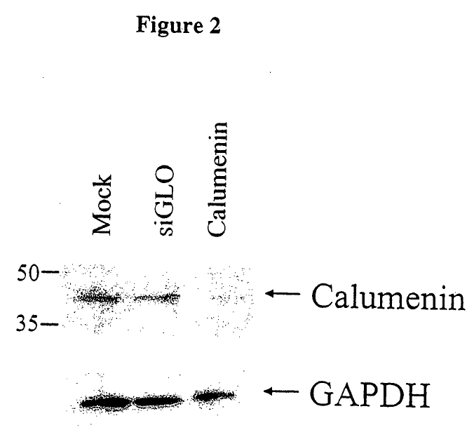

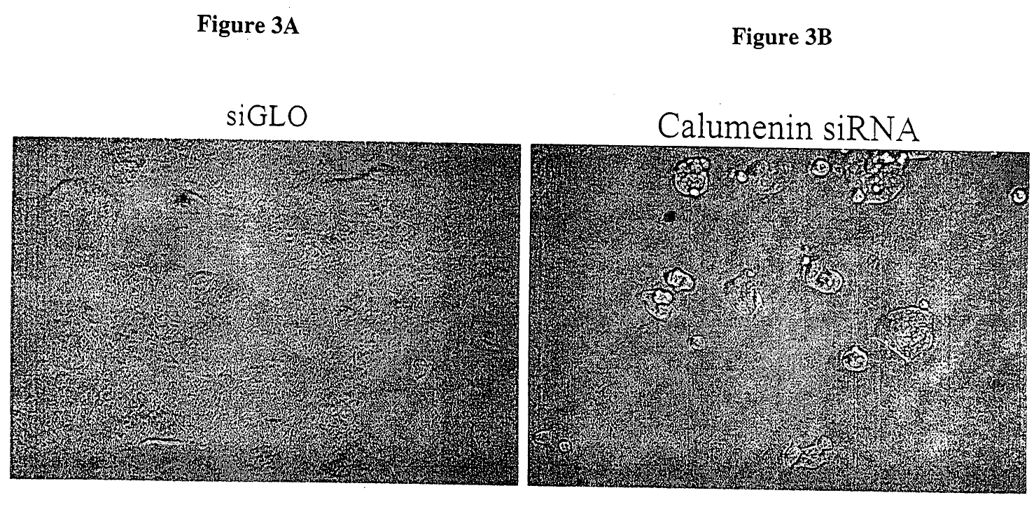

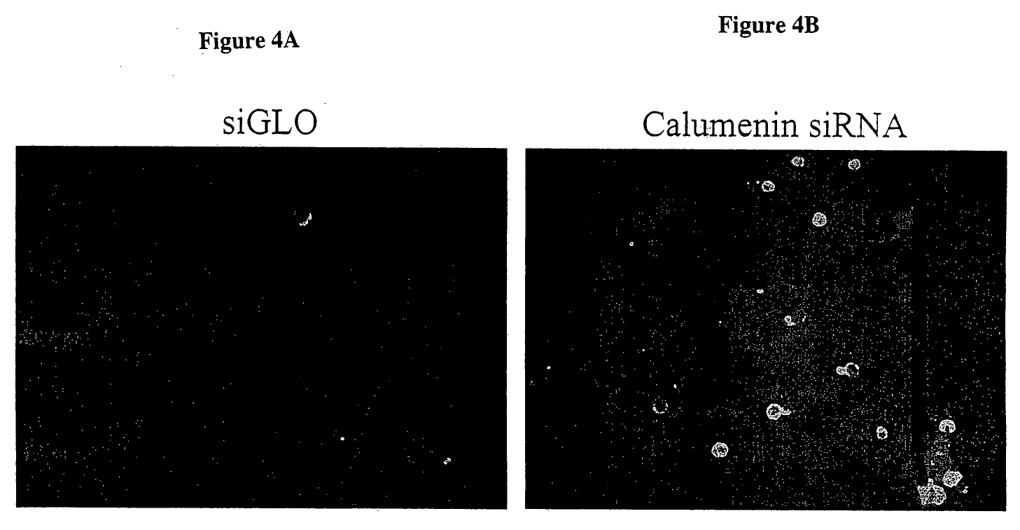

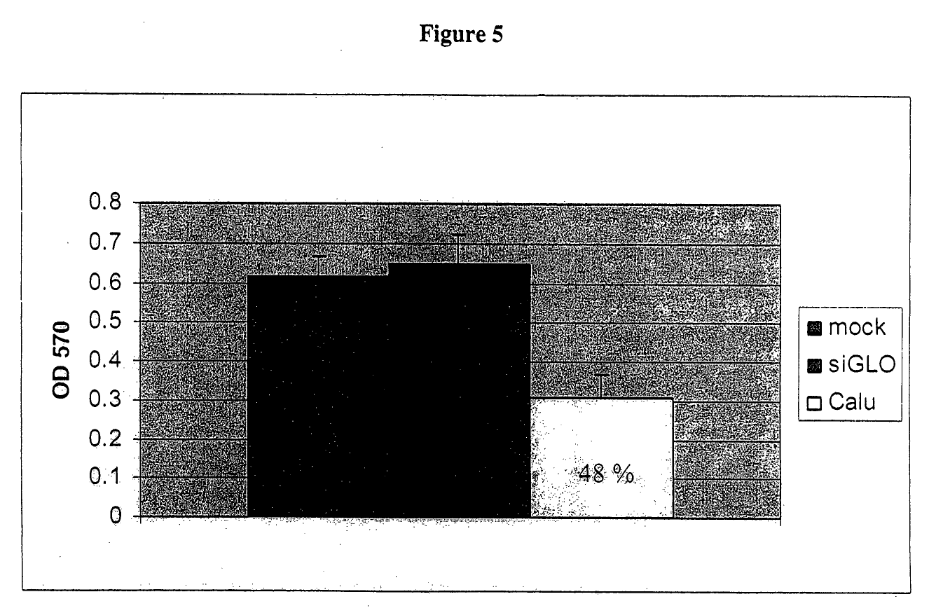

Both of these siRNA duplexes successfully down-regulated calumenin expression when compared to cells transfected with vectors expressing control siRNA sequences (FIG. 2). This decrease in calumenin expression was accompanied by a 50% decrease in cell viability of MCF-7 cells (FIG. 5). The calumenin-depleted cells also had a more rounded cell morphology (FIG. 3) characteristic of apoptotic cells, indicating that the calumenin depletion decreased the viability of the cells. The presence of apoptotic cells was confirmed by Annexin V staining. A substantially greater number of Annexin-V-positive cells were present in the calumenin siRNA transfected MCF-7 population as compared to the cell population treated with a control siRNA (FIG. 4). Similar effects on cell morphology and viability were observed with all calumenin specific siRNA duplexes as compared to control siRNAs.

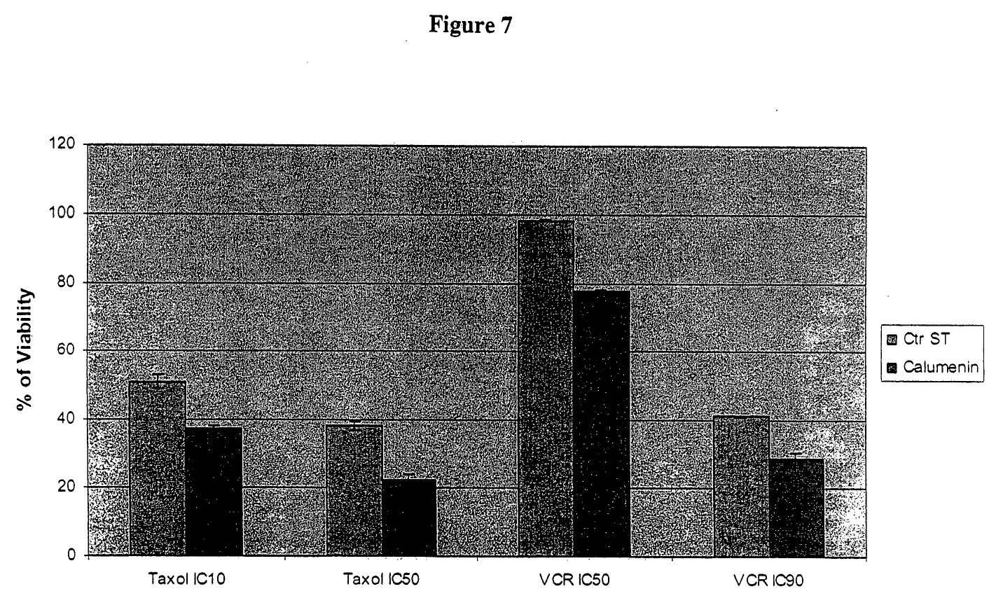

To evaluate the effect of calumenin depletion on the chemosensitivity of cells to cytotoxic agents, MTT assays were performed in MCF-7 cells transfected with calumenin-specific siRNAs. The calumenin silenced MCF-7 cells displayed a substantial increase in their sensitivity to taxol and vincristine as evidenced by the cytotoxicity graphs shown in FIG. 7. Cells transfected with calumenin-directed siRNA were 30% more sensitive to taxol at IC₁₀ drug treatment levels (FIG. 7). When taxol treatment was increased to IC₅₀ levels, the calumenin depleted cells were 50% more sensitive to taxol than cells transfected with control siRNA (FIG. 7). Likewise, calumenin depleted cells were between 20% and 33% more sensitive to vincristine at IC₅₀ and IC₉₀ drug treatment levels, respectively (FIG. 7).

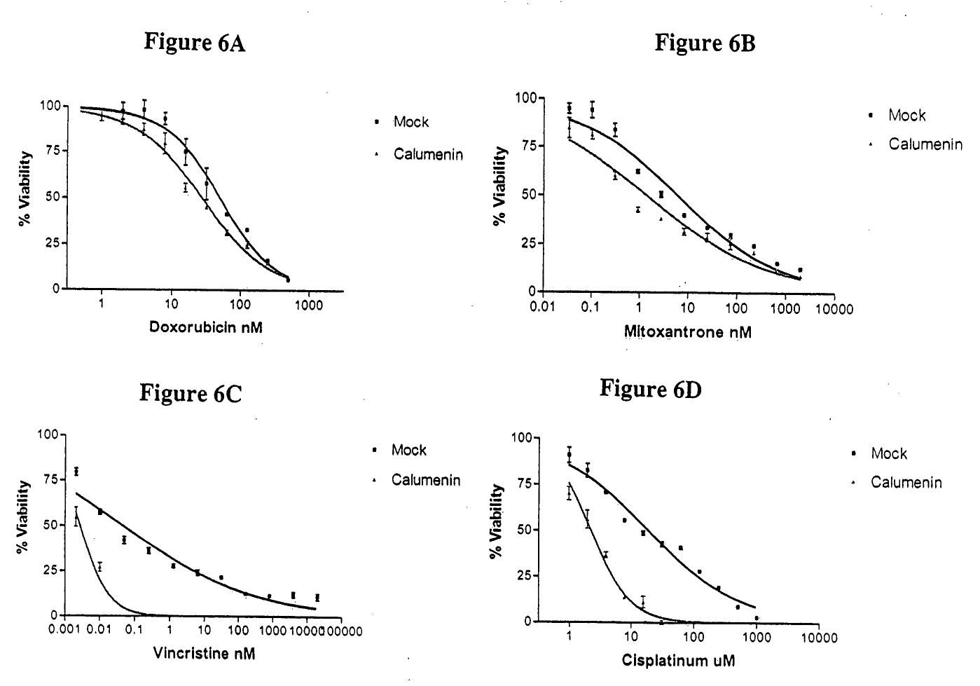

The EC₅₀ values for various chemotherapeutic drugs were determined for mock-transfected cells and cells transfected with calumenin-directed siRNA as well. Calumenin depletion decreased the EC₅₀ for all chemotherapeutic drugs when compared to mock transfected MCF-7 cells (FIGS. 6A-6D). MCF-7 cells became particularly sensitive to cisplatin and vincristine treatment, showing between 9 and 17 fold lower viability to drug treatment as compared to control cells (FIGS. 6C and 6D). Moreover, calumenin-depleted cells were two and four fold more sensitive to adriamycin and mitoxantrone, respectively (FIGS. 6A and 6B). The predicted EC₅₀ values for several chemotherapeutic drugs are shown in Table 2.

TABLE 2
Transfection of MCF7 Cells With Vector Expressing Calumenin

| Chemotherapeutic Drug | Control | | Calumenin | |
|---|---|---|---|---|
| Doxorubicin (nM) | 50.04 | (R2 = 0.9602) | 27.39 | (R2 = 0.9739) |
| | | | 1.8 × IS | |
| Cisplatin (μM) | 19.14 | (R2 = 0.9638) | 2.245 | (R2 = 0.5129) |
| | | | 8.5 × IS | |
| Taxol (nM) | 0.00002189 | (R2 = 0.5905) | 0.0000000053 | (R2 = 0.6932) |
| | | | 4130.2 × IS | |
| Etoposide (μM) | 1.2 | (R2 = 0.8742) | 0.9571 | (R2 = 0.9303) |
| | | | 1.3 × IS | |
| Mitoxantrone (nM) | 6.039 | (R2 = 0.9481) | 1.379 | (R2 = 0.9233) |
| | | | 4.4 × IS | |
| Docetaxel (nM) | 0.009764 | (R2 = 0.7828) | 0.004631 | (R2 = 0.8037) |
| | | | 2.1 × IS | |
| Vincristine (nM) | 0.04708 | (R2 = 0.9229) | 0.002735 | (R2 = 0.3147) |
| | | | 17.2 × IS | |
| Vinblastine (nM) | 0.001486 | (R2 = 0.8953) | 0.000002262 | (R2 = 0.8548) |

656.9 × IS

IS: “increased sensitivity” to the particular drug.
The EC₅₀ results were obtained 72 hours post transfection with either a vector expressing calumenin-encoding RNA or a mock vector.

Table 3 summarizes the results obtained in calumenin-depleted cell lines challenged with several chemotherapeutic drugs. In addition to the MCF-7 and MDA breast adenocarcinoma cell lines, the SKOV3 ovarian adenocarcinoma cell lines were transfected with calumenin-directed siRNA. The calumenin-depleted ovarian cancer cells showed similar decreases in viability as calumenin-depleted breast cancer cells (Table 3). Notably, the results from all tests on cancer cell lines established that calumenin-directed siRNA increases chemotherapeutic drug sensitivity by 1.6 to 17.2 fold as compared to mock siRNA-transfected cells (see Table 3).

TABLE 3
Summary of Results of Calumenin Silencing Experiments

| | Chemotherapeutic Drug | | | | |
|---|---|---|---|---|---|
| Cell | | Doxo- | | | |
| Line | Cisplatin | rubicin | Taxol | Vincristine | Mitoxantrone |
| MCF-7 | 8.5 × IS | 1.8 × IS | >10 × IS | 17 × IS | 4.4 × IS |
| MDA | NC | 2.4 × IS | 3.8 × IS | 3.8 × IS | 6 × IS |
| SKOV3 | 3.3 × IS | 1.6 × IS | 2.1 × IS | 4 × IS | 1.6 × IS |

IS: “increased sensitivity” to the particular drug.
NC indicates that no change occurred.

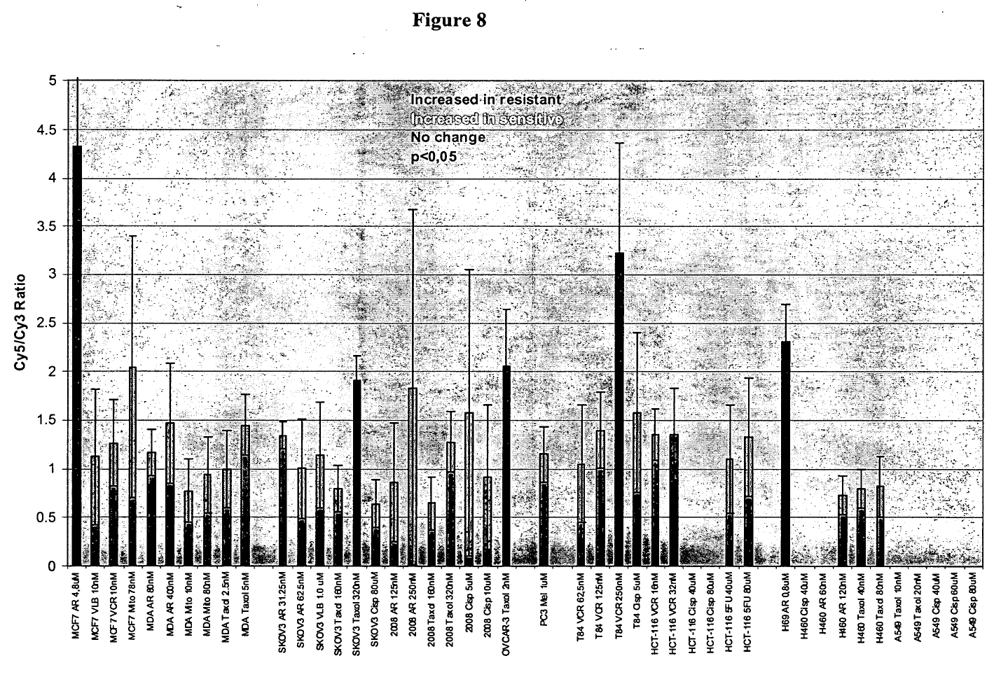

The expression levels of calumenin mRNA in chemotherapeutic drug-resistant cell lines was compared to calumenin expression levels in cell lines that were sensitive to chemotherapeutic drugs (FIG. 8). The taxol-resistant ovarian tumor cell line (OVCAR3) expressed calumenin at higher levels than its non-resistant control (FIG. 8). In addition, the vincristine-resistant colon carcinoma cell line (T84) and the adriamycin-resistant lung carcinoma cell line (H69) showed increased calumenin expression as compared to their respective non-resistant control cells (FIG. 8). These results indicate that calumenin mRNA levels can be used in some cancer cell types to predict chemotherapeutic drug resistance to certain drugs.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Overexpression of a 47 kD Protein in Cancer Cell Lines

Studies were performed to determine what proteins, if any, were differentially expressed in chemotherapeutic drug-resistant tumor cell lines as compared to their drug-sensitive counterparts. The nine different cell lines used in the Examples below are listed in Table 4.

TABLE 4

| Drug-Sensitive Cell Lines | Drug-Resistant Cell Lines |
|---|---|
| MCF-7 | MCF-7/AR |
| | MCF-7/VLB |
| | MCF-7/VCR |
| | MCF-7/Mito |
| | MDA/taxol |
| | MDA-MB-231/AR |
| | MDA/Mito |

Drug-sensitive control cell lines were obtained from were obtained from ATCC (Manassas, Va., USA). MCF7/AR was obtained from McGill University, Montreal, Qc, Canada. MDA-MB-231/AR was derived at Aurelium BioPharma Inc. (Montreal, QC, Canada). Additional chemotherapeutic drug-resistant cell lines used in the experiments were derived from a drug-sensitive clone of the “parent” cancer cell line representing a particular tissue.

All cell culture materials and reagents were obtained from Gibco Life Technologies (Burlington, Ont., Canada), or Sigma Chemical Corp. (St. Louis, Mo., USA) unless otherwise indicated.

Cells were cultured in a MEM medium supplemented with 10% fetal bovine serum (MCF7 and derivatives) or in DMEM high glucose medium supplemented with 10% fetal bovine serum (MDA-MB-231 and derivatives). All culture media contained L-glutamine (final concentration of 2 mM). The cells were grown in the absence of antibiotics at 37° C. in a humid atmosphere of 5% CO2 and 95% air. Chemotherapeutic drug-resistant cells (MCF-7/AR and MDA-MB-231/AR) were grown continuously with appropriate concentrations of cytotoxic drugs. All cell lines were examined for and determined to be free of mycoplasma contamination using a PCR-based mycoplasma detection kit according to manufacturer's instructions (Stratagene Inc., San Diego, Calif., USA). Chemotherapeutic drug-resistant cell lines were routinely tested for chemotherapeutic drug resistance using a panel of different drugs representing different classes of chemotherapeutic drugs.

Cell extracts from drug-resistant and drug-sensitive cell lines were prepared to determine the expression levels of potential therapeutic targets in drug-resistant cells. Briefly, cultured cells were rinsed 2 times with 15 ml of 1× phosphate buffered saline (“PBS”), and harvested by trypsinization. Cells were collected in a 15 ml tube by centrifugation at 1000 rpm for 5 min. The supernatant was discarded and cells were washed 3 times with 1×PBS. The cell pellet was transferred to an Eppendorf tube and 500 ml of 1×PBS were added. Cells were centrifuged 5 min. at 3000 rpm in an Eppendorf Microfuge. The supernatant was removed and cells were then lysed in 50 ml-150 ml of lysis buffer (50 mM Tris, pH 7.5, 150 mM NaCl, 1% NP-40, 0.5% sodium deoxycholate), containing protease inhibitors (1 mg/ml pepstatin, 1 mg/ml leupeptin, 1 mg/ml benzamidine, 0.2 mM PMSF) and incubated 5 min. on ice. The cell lysates were then centrifuged at 14,000×g for 10 min. at 4° C. The protein concentration of the supernatants was determined by the DC Protein assay (BioRad, Hercules, Calif.). Samples were subsequently stored at −80° C. until ready for analysis.

Total cell lysates were thawed and then incubated with 1 U/ml DNAse I (New England BioLabs, Inc., Beverly, Mass.), 5 mM MgCl₂ (final concentration) for 2 hours on ice. Their protein concentration was determined using the RC DC protein assay kit from BIORAD according to manufacturer's instructions (BioRad Laboratories, Hercules, Calif.) (see also Lowry et al., (1951) J. Biol. Chem. 193: 265-275). Equivalent amounts of proteins (250 mg) from total cell extracts from sensitive (MCF7, MDA-MB-231) and chemotherapeutic drug-resistant cells (MCF7/AR and MDA-MB-231/AR) were analyzed by polyacrylamide gel electrophoresis followed by blotting to nitrocellulose membrane. The membrane was subsequently contacted with an anti-calumenin antibody. The immunoblots were incubated with a secondary antibody and visualized using horseradish peroxidase using the manufacturer's protocol (Bio-Rad Laboratories, Hercules, Calif.).

The experiments identified a 47 kD protein that was of similar size to calumenin, and had significant reactivity to the anti-calumenin antibody (FIGS. 1A and 1B).

### Example 2

### Microarray Identification of Calumenin mRNA Overexpression

1. Total RNA Isolation and cDNA Labeling

Drug-resistant mRNA samples were isolated from cell lines (MCF-7, SKOV-3, MDA, 2008, OVCAR-3, PC3, T84, HCT-116, H69, and H460) that were resistant to various chemotherapeutic drugs. Cell lines can be obtained from ATCC (Rockville, Md.). The chemotherapeutic drugs used in the experiments included adriamycin (AR), vinblastinee (VLB), vincristine (VCR), mitoxantrone (Mito), taxol, cisplatin (Cisp), 5-FU, and melphalan (Mel). All drugs can be purchased from Sigma Corp. (St Louis, Mo.). Resistant cell lines and their sensitive counterparts were grown in cell specific medium conditions at 37° C./5% CO₂. Drug-sensitive cell samples were isolated from each cell line, and were used as control cell samples.

Cell lysis and RNA extraction was done with the RNEasy kit, (#74104) (Qiagen, Inc., Valencia, Calif.) following the manufacturer's protocol. RNA was quantified by spectrophotometry using a spectrophotometer (Ultrospec 2000, Amersham-Biosciences, Corp., Piscataway, N.J.). RNA samples were dissolved in 10 mM Tris, pH 7.5 to determine the A_{260/280} ratios. Samples with ratios between 1.9 and 2.3 were kept for probe preparation, while samples with ratios lower than 1.9 were discarded. RNA samples were dissolved in 1 μl DEPC-H₂O for total nucleic acid quantification. Total RNA from control and treated samples was dried by speed vacuum using a Heto Vacuum centrifuge system (KNF Neuberger, Inc., Trenton, N.J.) at varying time intervals. The total RNA was resuspended in 10 μl of DEPC-H₂O and stored at −20° C. until the labeling reaction.

First strand cDNA labeling was accomplished separately using 1-15 μg total RNA (depending on the cell lines to be tested) for the resistant and the sensitive cell lines. Total RNA was incubated with 4 ng control positive Arabidopsis thaliana RNA, 3 μg of Oligo (dT)₁₂₋₁₈ primer (#Y01212) (Invitrogen, Corp., Carlsbad, Calif.), 1 μg PdN6 random primer (Amersham, #272166-01) for 10 min. at 65° C., and immediately put on ice for 1 min. The mixture was then diluted in 5× First strand buffer (250 mM Tris-HCl, pH 8.3; 375 mM KCl; 15 mM MgCl₂) containing 0.1 M DTT, 0.5 μM dNTPs mix (dTTP, dGTP, dATP) (Invitrogen, #10297-018), 0.05 μM dCTP (Invitrogen, #10297-018), 5 μM Cy3-dCTP (#NEL 576) (NEN Life Science/Perkin Elmer, Boston, Mass.), 2.5 μM Cy5-dCTP (#NEL 577) (NEN Life Science/Perkin Elmer, Boston, Mass.) and 400 units SuperScript III RNAse H⁻ RT (Invitrogen, #18064-014). After incubating the reaction mixture for 5 min. at 25° C., the reaction mixture was incubated at 42° C. for 90 min. Finally, a total of 400 units of SuperScript II RNAse H⁻ RT (Invitrogen, #18064-014) were added and the reaction was incubated at 42° C. for another 90 min.

Digestion of the labeled cDNA with 5 units RNAse H (#M0297S) (NEB, Beverly, Mass.) and 40 units RNAse A (Amersham, #70194Y) was done at 37° C. for 30 min. The labeling probe was purified with the QIAquick PCR purification kit (Qiagen, Inc.) protocol with some modifications. Briefly, the reaction volume was completed to 50 μl with DEPC-H₂O and 2.7 μl of 12 M NaOAc pH 5.2 was added. The reaction was diluted with 200 μl PB buffer, put on the purification column, spun 15 sec. at 10 000 g, followed by 3 washes of 500 μl PE buffer (15 sec.; 10 000 g) and eluted 2 times in 50 μl DEPC-H₂O total (1 min.; 10 000 g). The frequency of incorporation and amount of cDNA labeled produced were evaluated for both labeled dCTPs by spectrophotometer (Ultrospec 2000, Pharmacia Biotech) at A_{260 nm}, A_{550 nm} and A_{650 nm}. The labeling material was dried by speed vacuum (Heto Vacuum centrifuge system, LaboPort) and resuspended in 3.75 μl H₂O total for both Cy5 (resistant cell line) and Cy3 reactions (sensitive cell line).

2. Capture Probe Preparation

Capture probes, approximately 68 nucleotides in length, including capture probes directed to calumenin, were designed using sequences showing less identity base to base (<30%) with other coding sequences (cds) submitted to NCBI bank. The comparisons between sequences were done by BLAST research (www.ncbi.nlm.nih.gov/BLAST). For BioChip ver1.0 and ver2.0, a basic melting point temperature at a salt concentration of 50 mM Na⁺ (Tm) for each capture probe was calculated: the overall average for all capture probes, including calumenin, was 76.97° C.+/−3.72° C. GC nucleotide content averaged 51.2%+/−9.4%. For the present invention, two negative controls (68 bp of the antisense cds of the BRCP and nucleophosmin targets) were synthesized.

The calumenin sequence present on the oligonucleotide array was derived from GenBank sequence (gi# 14718452) 880-943 bp of cds.

The capture probe was synthesized with the Expedilite™ Synthesizer at a coupling efficiency of over 99.5% (Applied Biosystems, Foster City, Calif.). All oligonucleotides on the microarray, including oligonucleotides directed to calumenin, were verified by polyacrylamide gel electrophoresis. Oligonucleotide quantification was done by spectrophotometry at A_{260 nm}.

3. Printing of Capture Probes and Production of the Focused Microarray

Prior to printing of capture probes, different dilutions of Arabidopsis thaliana chlorophyll synthetase G4 DNA (undiluted solutions at 0.15 μg/μl and at 0.2 μg/μl; 1:2; 1:4; 1:8; 1:16) were printed on each grid as a positive control, and for normalization of results. Preparation of Arabidopsis thaliana control capture probes was performed as follows. Briefly, 5 μg of a Midi preparation using a HiSpeed™ Plasmid Midi kit (Qiagen, Inc.) of the Arabidopsis thaliana plasmid (gift of BRI) was digested with 40 units of Sac I enzyme (NEB) for 2 hr. at 37° C., purified with the QIAquick PCR purification kit (Qiagen,) and verified by 1% agarose migration. In vitro transcription of 2 μg Sac I digestion was performed in 10× transcription buffer (400 mM Tris-HCl, pH 8.0; 60 mM MgCl₂; 100 mM DTT; 20 mM Spermidin) containing 2 μl of 10 mM NTP mix (Invitrogen), 20 units RNAse OUT (Invitrogen, #10777-019) and 50 units T7 RNA polymerase (NEB) for approximately 2 to 30 hr. at 37° C. The reaction was then treated with 2 units DNAse I (Invitrogen) in 10× DNAse buffer (200 mM Tris-HCI pH 8.4; 20 mM MgCl₂; 500 mM KCl) for 15 min. at 37° C. The RNA was cleaned with the RNEasy kit (Qiagen) and quantified by spectrophotometry using an Ultrospec 2000 (Amersham Biosciences, Corp.).

After the control capture probes, including capture probes directed to calumenin, were generated and printed, the capture probes complementary to marker genes from the cancer cell samples were printed at concentrations of 25 μM in 50% DMSO on CMT-GAPS II Slides (# 40003) (Corning, 45 Nagog Park, Acton, Mass.) by the VersArray CHIP Writer Prosystems (BioRad Laboratories) with the Stealth Micro Spotting Pins (#SMP3) (Telechem International, Inc., Sunnyvale, Calif.). Each capture probe, including probes directed to calumenin, was printed in triplicate on duplicate grids. Buffer and Salmon Testis DNA (Sigma D-7656) were also printed for the BioChip analysis step. After printing was completed, the slides were dried overnight by incubation in the CHIP Writer chamber. Chips were then treated by UV (Stratagene, UV Stratalinker) at 600 mJoules and baked in an oven for 6-8 hr.

4. Quality Control of Focused Microarray

Prior to testing the invention on cancer cell samples, the focused microarray was tested at the BRI Institute (Kowloon Bay, Hong Kong). One slide for each printed batch was quality control tested using a terminal deoxynucleotidyl transferase (Tdt)-mediated nick end labeling assay protocol (see, e.g., Yeo et. al., (2004) Clin. Cancer Res. 10(24): 8687-96). Additionally, controls were performed to verify the specificity of the hybridization using three independent grids on the same focused microarray.

As a first quality control, a test was done by the BRI Institute on one slide for each batch printed with the following Tdt transferase protocol. Briefly, the slide was prehybridized in a Hybridization Chamber (#2551) (Corning, Inc., Life Sciences, 45 Nagog Park, Acton, Mass.) with 80 μl of preheated prehybridization buffer (5×SSC (750 mM NaCl; 75 mM sodium citrate); 0.1% SDS; 1% BSA (Sigma, #A-7888) at 37° C. for 30 min. Slides were washed in 0.1×SSC (15 mM NaCl; 1.5 mM sodium citrate) and air-dried. Fifty micro-liters of TdT reaction mixture [5× TdT buffer (125 mM Tris-HCl, pH 6.6, 1 M sodium cacodylate, 1.25 mg/ml BSA); 5 mM CoCl₂; 1 mM Cy3-dCTP (NEN Life Science, NEL 576); 50 units TdT enzyme (#27-0730-01) (Amersham BioSciences)], was added to the entire area of the BioChip. The slide was incubated in the Hybridization Chamber for 60 min. at 37° C. following by a first wash in 1×SSC (150 mM NaCl; 15 mM sodium citrate)/0.2% SDS (preheated at 37° C.) for 10 min., a second wash of 5 min. in 0.1×SCC (15 mM NaCl; 1.5 mM sodium citrate)/0.2% SDS at room temperature and finally a last wash of 5 min. at room temperature in 0.1×SSC (15 mM NaCl; 1.5 mM sodium citrate). The slide was scanned with the ScanArray™ Lite MicroArray Scanner (Packard BioSciences, Perkin Elmer, San Jose, Calif.).

As a second quality control step, the PARAGON™ DNA Microarray Quality Control Stain kit (Molecular Probes) was incubated with the microarray according to the manufacturer's recommendations.

5. Focused Microarray Hybridization with Labeled cDNA Probes

Focused microarray slides were pre-washed before the prehybridization step as follows. First, slides were washed for 20 min. at 42° C. in 2×SSC (300 mM NaCl; 30 mM sodium citrate)/0.2% SDS under agitation. The second wash was for 5 min. at room temperature in 0.2×SSC (30 mM NaCl, 3 mM Sodium citrate) under agitation, and then followed by a wash for 5 min. at room temperature in DEPC-H₂O with agitation. The slides were spin dried at 1000 g for 5 min. and prehybridized in Dig Easy Hyb Buffer (#1,603,558) (Roche Diagnostics Corporation, Indianapolis, Ind.) containing 400 μg Bovine Serum Albumin (Roche, #711,454) at 42° C. in humid chamber for 3 hr. then washed 2 times in DEPC-H₂O, and once in Isopropanol (Sigma, 1-9516) and spun dry at 1000 g for 5 min.

To the mixed Cy5/Cy3 probe, 15 μg Baker tRNA (#109,495) (Roche Diagnostics Corp., Indianapolis, Ind.) and 1 μg Cot-1 DNA (Roche, #1,581,074) were added and the probe was incubated 5 min. at 95° C., put on ice for 1 min., and diluted with 14 μl Dig Easy Hyb buffer (Roche, #1,603,558). After a 2 min. spin at 100×g, the probe was incubated at 42° C. for at least 5 min.

The three supergrids on the slide were separated by a Jet-Set Quick Dry TOP Coat 101 line (#FX268) (L'Oreal, Paris, FR) (FIGS. 1A-1C). Each probe was added to its respective supergrid and covered by a preheated (42° C.) coverslip (Mandel, #S-104 84906). The slide was incubated at 42° C. in humid chamber for at least 15 hr.

The coverslips were removed by dipping in 1×SSC (150 mM NaCl; 15 mM sodium citrate)/0.2% SDS solution preheated at 50° C.). The slide washed three times for 5 min. with agitation in 1×SSC (150 mM NaCl; 15 mM sodium citrate)/0.2% SDS solution preheated at 50° C.), and then washed three times with agitation in 0.1×SSC (15 mM NaCl; 1.5 mM sodium citrate)/0.2% SDS solution preheated at 37° C.). Finally, the slide washed once in 0.1×SSC (15 mM NaCl; 1.5 mM sodium citrate) with agitation for 5 min. The slide was dipped several times in DEPC-H₂O and spun dry at 1000 g for 5 min.

6. Scanning and Statistical Analysis

The slides were scanned with a ScanArray™ Lite MicroArray Scanner (Packard BioSciences, Perkin Elmer, San Jose, Calif.) and the analysis was performed with a QuantArrayR Microarray Analysis software version 3.0 (Packard BioSciences, Perkin Elmer, San Jose, Calif.).

The QuantArray® data results were analyzed according to the following procedures. All analysis of the results was performed with the spot background subtracted values for Cy5 and Cy3. Spots with lower signal ratio to noise lower than 1.5 were discarded. Normalization of the ratios with the spike positive control (Arabidopsis thaliana) was done to have a ratio equal to one for that control on each slide. Slides were discarded on which the negative and/or positive controls did not work. Also, slides were discarded with high background and with different mean no offset correction (ArrayStat software). Mean for each target was calculated with at least six different experiments (including two reciprocal labeling reactions), each experiment using different total RNA preparations. Statistical analysis was accomplished with the ArrayStat 1.0 (Imaging Research Inc., Brock University, St. Catherine's, Ontario, Calif.). A log transformation of the ratio data is followed by a Student T test for two independent conditions using a proportional model without offsets at a p<0.05 threshold. Significant increases (ratio Cy5/Cy3 higher than 1.5) or decreases (ratio Cy5/Cy3 lower than 0.5) were considered to be significant if the p value was lower than 0.05.

7. Results.

Microarray analysis established that calumenin mRNA was expressed at higher levels in certain drug-resistant cell lines as compared to cell lines sensitive to the same drug (FIG. 8). In particular, MCF-7, SKOV3, OVCAR3, and T84 cell lines showed statistically significant increases in calumenin mRNA expression as compared to cell lines that were sensitive to the sam drug (FIG. 8).

### Example 3

### Targeted Silencing of Calumenin in Cell Lines

To establish the importance of calumenin to the expression of the drug-resistant phenotype in MCF-7 cell lines, calumenin expression was silenced using RNAi. Briefly, the following siRNA duplexes targeting the human calumenin mRNA were designed and purchased either from Ambion (Austin, Tex.) or Invitrogen (Carlsbad, Calif.). The siRNA duplex sequences corresponding to nucleotides 343-352 (REFSEQ ID NUMBER: NM_{—}001428) targeting the start of the calumenin mRNA transcript were:

sense strand 5′-GAAGGACCGUGUACAUCAUtt-3′ (SEQ ID NO: 1);

anti-sense strand: 5′-AUGAUGUACACGGUCCUUCtt-3′ (SEQ ID NO: 2). The siRNA duplex was predesigned, synthesized with 3′TT overhangs, purified and annealed by Ambion (Austin, Tex.).

Two chemically modified Stealth™ RNAi duplexes targeting calumenin were designed using the Block-it™ RNAi Designer tool by Invitrogen (http://rnaidesigner.invitrogen.com/sima/design.do). The corresponding duplexes TARGETING NUCLEOTIDES 337-352 OF THE CALUMENIN mRNA were:

Calu-1 sense strand

(SEQ ID NO:3)

5′-GGGUGCUGAAGAAGCAAAGACCUUU-3′
and
anti-sense strand

(SEQ ID NO:4)

5′-AAAGGUCUUUGCUUCUUCAGCACCC-3′.

As a negative control, the following scrambled sequence not having significant homology to any human gene was designed: 5′-CCAGGGUUCCUAAUCGGAUUUGCUA-3′ (SEQ ID NO: 5). The siRNA duplex targeting VEGF was the chemically modified Stealth version of the following duplexes:

sense strand

(SEQ ID NO:8)

5′-ACAAAUGUGAAUGCAGACCAAAGAA-3′;
anti-sense strand

(SEQ ID NO:9)

5′-UUCUUUGGUCUGCAUUCACAUUUGU-3′

(Filleur et al. (2003) Cancer Res. 63(14): 3919-22). All above duplexes were synthesized, purified and annealed by the manufacturer (Invitrogen). To monitor transfection efficiency, a Cy3-labeled GL2 siRNA duplex against firefly luciferase was purchased from Dharmacon, Inc. (Chicago, Ill.). For the chemically modified Stealth siRNA's, the non-targeting siGLO™ fluorescent siRNA duplex (Dharmacon, Chicago, Ill.) or the Block-it™ Fluorescent oligonucleotide (Invitrogen, Carlsbad, Calif.) was used. Transfection efficiencies were typically evaluated 24-48 hrs post transfection using a fluorescence microscope. The levels achieved were routinely greater than 95%.

For a typical siRNA transfection, 1 mmole of the annealed siRNA duplex was mixed with 1.4 ml of Opti-MEM reagent (Invitrogen). In another tube, 85 ml of Oligofectamine reagent (Invitrogen, Carlsbad, Calif.) was mixed with 600 ml of Opti-MEM. The two solutions were combined and mixed gently by inversion and incubated for 20 min. at RT. The resulting solution was added to the cultured cells drop by drop in a 10 cm dish (cells are approximately 40-50% confluent). The next day the transfected cells were trypsinized and seeded in 6 or 96-well plates and further incubated for the indicated amount of time (assay dependent) before further analysis. Silencing efficacy results are shown in FIG. 2.

To determine the ability of cells to proliferate after they were transfected with a vector containing the coding sequences for the calumenin gene or control siRNAs as indicated above, the cells were seeded the next day in a 96-well plate at 5×10³ cells/well in quadruplicate. The plate was incubated at 37° C. incubator for another 72 hrs. The media was removed, and 100 ml of CyQUANT GR dye/cell lysis buffer (Molecular Probes, Inc., Eugene, Oreg.) was added per well. The plate was incubated for 5 min. at RT in the dark. The resulting fluorescence was measured in a Wallac microplate reader (PerkinElmer, Inc., Boston, Mass.) using a 535 nm filter. Results were the average of quadruplicates and were plotted in Excel. The number of cells was determined by extrapolation from a standard curve. FIG. 2 shows the overall effects of calumenin siRNA treatment on the levels of expression of calumenin in MCF-7 cells. Calumenin siRNA significantly decreased calumenin expression as compared to mock and siGLO cells (FIG. 2).

### Example 4

### Effects of Calumenin Silencing on Cell Survival

1. MTT Cytotoxicity Assay

Cell survival was determined using the MTT cytotoxicity assay (see, e.g., Tokuyama et al. (2005) Anticancer Res. 25(1A): 17-22). Small interfering RNA transfected cells were seeded in triplicate into 96-well plates at 5×10³ cells/well 48 hrs post-transfection. The cells were incubated for an additional 16 to 24 hrs before they were exposed to increasing concentrations of cytotoxic drugs. Doxorubicin (adriamycin), cisplatin, taxol, vinblastine, vincristine, and mitoxantrone were all purchased from Sigma Corp. (St. Louis, Mo.). Stocks were made as follows: 6 mM for doxorubicin, 1.1 mM for vincristine and vinblastine; 1.1 mM for taxol, 50 mM for cisplatin both in DMSO; and 0.97 mM mitoxantrone in ethanol. Appropriate dilutions were made in the respective media for each cell line. Following addition of drugs, incubation was continued for an additional 72 hrs. Twenty-five ml of MTT dye (5 mg/ml) were added into each well and the plate was further incubated at 37° C. for 4 hrs. The dye was solubilized with 10% Triton X-100, 0.01 N HCl and further incubated at 37° C. in the dark for 30 min. Cell viability was determined by measure of absorption at 570 nm in a Wallac multiwell plate reader (PerkinElmer, Inc., Boston, Mass.). The averages of triplicate wells were plotted using the Prism software (GraphPad Software, Inc., San Diego, Calif.).

The results indicate that MCF-7 cells had decreased viability when treated with chemotherapeutic drugs in combination with calumenin siRNA (FIGS. 6A-6D). FIG. 5 also shows that cells expressing calumenin-directed siRNA were 48% less viable than mock transfectants and siGLO control siRNA transfectants.

2. Clonogenic Assay

A clonogenic assay generated additional information concerning cell viability after drug-resistant cells were exposed to siRNA and chemotherapeutics. Briefly, transfected MCF-7 cells were seeded in triplicate into 24-well plates at 5×10³ cells/well 48 hrs post-transfection. The cells were incubated for an additional 16-24 hrs before they were exposed to increasing concentrations of cytotoxic drugs. Taxol or Vincristine was added at IC₁₀ or IC₅₀ concentrations determined from MTT experiments for MCF-7 cells. The IC₁₀ for taxol was 1 nM and IC₅₀=100 nM; for vincristine, IC₁₀ and IC₅₀ were determined to be 5 μM and 0.25 nM, respectively. The cells were further incubated for an additional 7 days. At the end of the incubation, the cells were stained with addition of a 0.5% Methylene Blue solution in 50% ethanol for 15 min. at RT. The staining solution was then removed and plates were dried overnight. The plates were scanned and the stained colonies were solubilized in 0.1% SDS. The absorbance of the resulting solution was determined by spectrophotometry at 660 nm. Results were plotted as bar graphs as shown in FIG. 7. FIG. 7 shows that at IC₁₀ and IC₉₀ taxol concentrations, calumenin-directed siRNA and taxol treatment significantly decreased cell survival as compared to taxol treatment alone. The same results were found for cells treated with vincristine and calumenin-directed siRNA as compared to vincristine alone (FIG. 7).

3. Apoptosis Assays

This assay was performed to determine the number of cells that were susceptible to chemotherapeutic drugs following incubation with siRNAs. Cells transfected with siRNA were seeded in Lab-Tek 16-well chamber slides (Electron Microscopy Sciences, Hatfield, Pa.) at 10⁴ cells/well 48 hrs post-transfection. Apoptosis was determined 16 hours later by annexin-V staining using the Annexin-V FLUOS kit (Roche, Ltd., Basel, CH) following the manufacturer's instructions. Slides were observed under a fluorescence microscope and images were taken using an Olympus digital camera and the Q-Capture software (QImaging, Burnaby, BC, CA).

Cell survival was decreased in cells treated with calumenin-directed siRNA as compared to cells treated with siGLO control siRNA (FIG. 4).

### Example 5

### Calumenin-Targeted Therapy Against MDR Cancer Cells

1. Treatment of MDR Hematological Cancer Cells

In order to determine whether targeting calumenin is useful in treating a preexisting cancerous condition, MHC-matched mice, 5 to 7 weeks old, receive a subcutaneous (s.c.) injection of 5×10⁵ hematological tumor cells, and tumors are allowed to form. Tumor growth starting on the first day of treatment is measured by palpitation, and the volume of the xenograft is monitored every 4 days. Tumors are allowed to grow to a sufficient size (5.5 mm) for appropriate analysis of the effects of calumenin treatment on tumor sensitivity to chemotherapeutic drugs. Mice are then treated with a calumenin siRNA (3 μg daily for 16 days) designed to decrease the level of expression of calumenin. Control mice receive no treatment, treatment with taxol or doxorubicin alone (4 mg/kg daily) or treatment with control siRNA sequences that are not complementary to murine calumenin mRNA (3 μg daily for 16 days for each treatment) in combination with taxol or doxorubicin (4 mg/kg daily). Taxol and doxorubicin can be obtained commercially from Sigma Corp. (St. Louis, Mo.).

Treatment with siRNA specific for calumenin mRNA sequences increases the sensitivity of hematological tumors to chemotherapeutic drug treatment regimes. As a result, the mice that receive the composition show a better prognosis (i.e., smaller tumor or fewer tumor cells) as compared to mice that receive only the targeting agent or only the taxol or doxorubicin.

Control siRNA sequences are utilized that do not represent binding sequences to murine calumenin (3 μg daily for 16 days for each treatment). The animal's weight is measured every 4 days. Tumor growth starting on the first day of treatment is measured by palpitation and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the calumenin siRNA and chemotherapeutic drugs are compared to tumor weights from mice treated with control siRNA and chemotherapeutic drugs. Tumor cell count is determined by trypsinizing tumors in DMEM medium supplemented with 10% fetal bovine serum until cells are in free suspension. Cells are then transferred to 6-well plates for counting. Cell counts are compared. All experiments are performed in triplicate.

2. Treatment of Mammary Adenocarcinoma

In further studies, the efficacy of a calumenin-targeted therapeutic in treating an MDR mammary adenocarcinoma cells (MCF/AR) is assessed. Briefly, male thymic nude mice 5 to 7 weeks old, weighing 18 g to 22 g, are used for the MCF-7/ADR xenografts. Mice receive a subcutaneous (s.c.) injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, calumenin siRNA alone (3 μg daily for 16 days), or both taxol and calumenin siRNA (3 μg daily for 16 days for each treatment). Control siRNA sequences are utilized that do not represent binding sequences to murine calumenin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the calumenin siRNA and chemotherapeutic drugs are compared to tumor weights from mice treated with control siRNA and chemotherapeutic drugs. Cell counts are compared. All experiments are performed in triplicate.

For both multidrug-resistant hematological cancers and adenocarcinomas, mice treated with the calumenin-directed siRNA have smaller tumors by weight than mice treated with control siRNA. In addition, total cell numbers of tumors isolated from mice treated with calumenin siRNA are lower than mice treated with control siRNA.

### Example 6

### Calumenin Targeted Therapy Against Cancer Cells

1. Treatment of Hematological Tumors

In order to determine whether targeting calumenin is useful in treating a preexisting cancerous condition, MHC-matched mice, 5 to 7 weeks old, receive an s.c. injection of 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of calumenin treatment on tumor sensitivity to chemotherapeutic drugs. Mice are then treated with a calumenin siRNA (3 μg daily for 16 days) designed to decrease the level of expression of calumenin. Control mice receive no treatment, treatment with taxol or doxorubicin alone (4 mg/kg daily) or treatment with control siRNA sequences that are not complementary to murine calumenin mRNA (3 μg daily for 16 days for each treatment) in combination with taxol or doxorubicin (4 mg/kg daily). Taxol and doxorubicin can be obtained commercially from Sigma Corp. (St. Louis, Mo.).

Control siRNA sequences are utilized that do not represent binding sequences to murine calumenin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the calumenin siRNA and chemotherapeutic drugs are compared to tumor weights from mice treated with control siRNA and chemotherapeutic drugs. Tumor cell count is determined by trypsinizing tumors in DMEM medium supplemented with 10% fetal bovine serum until cells are in free suspension. Cells are then transferred to 6-well plates for counting. Cell counts are compared. All experiments are performed in triplicate.

2. Treatment of Mammary Adenocarcinoma

In further studies, the efficacy of a calumenin-targeted therapeutic in treating a mammary adenocarcinoma cells (MCF-7) is assessed. Briefly, male thymic nude mice 5 to 7 weeks old, weighing 18 g to 22 g, are used for the MCF-7/ADR xenografts. Mice receive an s.c. injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, calumenin siRNA alone (3 μg daily for 16 days), or both taxol and calumenin siRNA (3 μg daily for 16 days for each treatment). Control siRNA sequences are utilized that do not represent binding sequences to murine calumenin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the calumenin siRNA and chemotherapeutic drugs are compared to tumor weights from mice treated with control siRNA and chemotherapeutic drugs.

For both multidrug-resistant hematological cancers and adenocarcinomas, mice treated with the calumenin-directed siRNA have smaller tumors by weight than mice treated with control siRNA. In addition, total cell number in tumors isolated from mice treated with calumenin-directed siRNA is lower than mice treated with control siRNA.

### Example 7

### Calumenin Liposome Formulation for Targeted Therapy Against Cancer Cells

1. Treatment of Hematological Cancer

In order to determine whether targeting calumenin is useful in treating a preexisting cancerous condition, MHC-matched mice, 5 to 7 weeks old, receive an s.c. injection of 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of calumenin treatment on tumor sensitivity to chemotherapeutic drugs. Mice are then treated with a liposome formulation containing calumenin siRNA designed to decrease the level of expression of calumenin.

Liposome formulations are produced as described previously (Shi et al. (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Calumenin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

The liposome treatment introduces 3 μg of calumenin-targeted siRNA per day for 16 days. Control mice receive no treatment, treatment with taxol or doxorubicin alone (4 mg/kg daily) or treatment with liposomes containing control siRNA sequences that are not complementary to murine calumenin mRNA (3 μg daily for 16 days for each treatment) in combination with taxol or doxorubicin (4 mg/kg daily). Taxol and doxorubicin can be obtained commercially from Sigma Corp. (St. Louis, Mo.).

The cancer cells treated with liposome/calumenin siRNA treatment show an increase in sensitivity to chemotherapeutic treatment regimes. As a result, the mice that receive the composition show a better prognosis (i.e., smaller tumor or fewer tumor cells) as compared to mice that receive only the targeting agent or only the vincristine.

A determination of decreased tumor size or cancer cell number is made by sacrificing the mice and excising the tumor. The size of the tumor in mice treated with the calumenin targeting agent and chemotherapy is measured and compared to measurements obtained from tumors in mice treated with chemotherapy alone. Tumor cell count is determined by trypsinizing tumors in DMEM medium supplemented with 10% fetal bovine serum until cells are in free suspension. Cells are then transferred to six well plates for counting. Cell counts are compared. All experiments are performed in triplicate.

2. Treatment of Mammary Adenocarcinoma

In further studies, the efficacy of a calumenin-targeted therapeutic in treating a mammary adenocarcinoma cells (MCF-7) is assessed. Briefly, male thymic nude mice 5 to 7 weeks old, weighing 18 g to 22 g, are used for the MCF-7/ADR xenografts. Mice receive an s.c. injection of the cells using 5×10⁵ cells/inoculation under the shoulder.

Liposome formulations are produced as described previously (Shi et al. (2000) Proc. Natl. Acad. Sci. USA. 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and are sonicated for 10 min. Calumenin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

When the s.c. tumor is approximately 5.5 mm in size, mice are randomized into treatment groups of 4 including controls and groups receiving taxol or doxorubicin, alone (4 mg/kg), intraperitoneally (i.p.) every 2 days, calumenin siRNA/liposome formulation alone (3 μg daily for 16 days), or both taxol and calumenin siRNA/liposome formulation (3 μg daily for 16 days for each treatment). Control siRNA sequences are utilized that do not represent hybridizing sequences to murine calumenin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the calumenin siRNA and chemotherapeutic drugs are compared to tumor weights from mice treated with control siRNA and chemotherapeutic drugs.

For both multidrug-resistant hematological cancers and adenocarcinomas, mice treated with the liposome formulations containing calumenin-directed siRNA have smaller tumors by weight than mice treated with liposome formulations containing control siRNA. In addition, total cell number in tumors isolated from mice treated with liposome formulations containing calumenin-directed siRNA is lower than mice treated with liposome formulations containing control siRNA.

### Example 8

### Calumenin Immunoliposome Formulation for Targeted Therapy Against Cancer Cells

1. Treatment of Hematological Cancer

In order to determine whether targeting calumenin is useful in treating a preexisting cancerous condition, MHC-matched mice, 5 to 7 weeks old, receive an s.c. injection of 5×10⁵ hematological tumor cells, and tumors are allowed to form. Tumor growth starting on the first day of treatment is measured by palpitation and the volume of the xenograft is monitored every 4 days. Tumors are allowed to grow to a sufficient size (5.5 mm) for appropriate analysis of the effects of calumenin treatment on tumor sensitivity to chemotherapeutic drugs. Mice are then treated with an immunoliposome formulation containing calumenin siRNA designed to decrease the level of expression of calumenin.

Immunoliposome formulations are produced as described by Shi et al. (Proc. Natl. Acad. Sci. USA. (2000) 97(13): 7567-7572). Briefly, POPC (19.2 μmol), DDAB (0.2 μmol), DSPE-PEG 2000 (0.6 mol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and sonicated for 10 min. Calumenin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm; and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

An anti-nucleophosmin mAb is obtained commercially, or is harvested, from serum-free nucleophosmin hybridoma-conditioned media. The anti-nucleophosmin mAb, as well as the isotype control, mouse IgG2a, are purified by protein G Sepharose affinity chromatography. The anti-nucleophosmin mAb or mouse IgG2a (1.5 mg, 10 nmol) is thiolated by using a 40:1 molar excess of 2-iminothiolane (Traut's reagent), as described by Huwyler et al. (Proc. Natl. Acad. Sci. USA. (1996) 93:14164-14169). Thiolated mAb is conjugated to pegylated liposomes using standard procedures also described by Huwyler et al. (Proc. Natl. Acad. Sci. USA. (1996) 93:14164-14169). This preparation is then administered to the animals.

The immunoliposome treatment introduces 3 μg of calumenin-targeted siRNA per day for 16 days. Control mice receive no treatment, treatment with taxol or doxorubicin alone (4 mg/kg daily) or treatment with liposomes containing control siRNA sequences that are not complementary to murine calumenin mRNA (3 μg daily for 16 days for each treatment) in combination with taxol or doxorubicin (4 mg/kg daily). Taxol and doxorubicin can be obtained commercially from Sigma Corp. (St. Louis, Mo.).

The cancer cells treated with the immunoliposome/calumenin siRNA treatment show an increase in sensitivity to chemotherapeutic treatment regimes. As a result, the mice that receive the composition show a better prognosis (i.e., smaller tumor or fewer tumor cells) as compared to mice that receive only the targeting agent or only the vincristine.

A determination of decreased tumor size or cancer cell number is made by sacrificing the mice and excising the tumor. The size of the tumor in mice treated with the calumenin targeting agent and chemotherapy is measured and compared to measurements obtained from tumors in mice treated with chemotherapy alone. Tumor cell count is determined by trypsinizing tumors in DMEM medium supplemented with 10% fetal bovine serum until cells are in free suspension. Cells are then transferred to six well plates for counting. Cell counts are compared. All experiments are performed in triplicate.

2. Treatment of Adenocarcinoma

In further studies, the efficacy of a calumenin-targeted therapeutic in treating a mammary adenocarcinoma cells (MCF-7) is assessed. Briefly, male thymic nude mice 5 to 7 weeks old, weighing 18 g to 22 g is used for the MCF-7/ADR xenografts. Mice receive an s.c. injection of the cells using 5×10⁵ cells/inoculation under the shoulder. When the s.c. tumor is approximately 5.5 mm in size, mice are randomized into treatment groups of 4 including controls and groups receiving taxol or doxorubicin, alone (4 mg/kg), intraperitoneally (i.p.) every 2 days, calumenin siRNA/immunoliposome formulation alone (3 μg daily for 16 days), or both taxol and calumenin siRNA/immunoliposome formulation (3 μg daily for 16 days for each treatment).

Immunoliposome formulations are produced as described by Shi et al. (Proc. Natl. Acad. Sci. USA. (2000) 97(13): 7567-7572). Briefly, POPC (19.2 μmol), DDAB (0.2 μmol), DSPE-PEG 2000 (0.6 μmol), and DSPE-PEG 2000-maleimide (30 nmol) are dissolved in chloroform/methanol (2:1, vol:vol) after a brief period of evaporation. The lipids are dispersed in 1 ml 0.05 M Tris-HCl buffer, pH 8.0, and sonicated for 10 min. Calumenin siRNA is added to the lipids. The liposome/siRNA dispersion is evaporated to a final concentration of 200 mM at a volume of 100 μl. The dispersion is frozen in ethanol/dry ice for 4 to 5 min. The dispersion is then thawed at 40° C. for 1 to 2 min, and this freeze-thaw cycle is repeated 10 times. The liposome dispersion is diluted to a lipid concentration of 40 mM, is followed by extrusion 10 times each through two stacks each of 400 nm, 200 nm, 100 nm, and 50 nm pore size polycarbonate membranes, by using a hand held extruder (Avestin, Ottawa). The mean vesicle diameters are determined by quasielastic light scattering using a Microtrac Ultrafine Particle Analyzer (Leeds-Northrup, St. Petersburg, Fla.).

An anti-nucleophosmin mAb is obtained commercially, or is harvested from serum-free nucleophosmin hybridoma-conditioned media. The anti-nucleophosmin mAb, as well as the isotype control, mouse IgG2a, are purified by protein G Sepharose affinity chromatography. The anti-nucleophosmin mAb or mouse IgG2a (1.5 mg, 10 nmol) is thiolated by using a 40:1 molar excess of 2-iminothiolane (Traut's reagent), as described by Huwyler et al. (Proc. Natl. Acad. Sci. USA. (1996) 93:14164-14169). Thiolated mAB is conjugated to pegylated liposomes using standard procedures also described by Huwyler et al. (Proc. Natl. Acad. Sci. USA. (1996) 93:14164-14169). This preparation is then administered to the animals.

Control siRNA sequences are utilized that do not represent binding sequences to murine calumenin (3 μg daily for 16 days for each treatment). The animal's weight is measured every 4 days. Tumor growth starting on the first day of treatment is measured and the volume of the xenograft is monitored every 4 days. The mice are anaesthetized and sacrificed when the mean tumor weight is over 1 g in the control group. Tumor tissue is excised from the mice and its weight is measured. Tumor weights from mice treated with the calumenin siRNA and chemotherapeutic drugs are compared to tumor weights from mice treated with control siRNA and chemotherapeutic drugs.

For both multidrug-resistant hematological cancers and adenocarcinomas, mice treated with immunoliposome formulations containing calumenin-directed siRNA have smaller tumors by weight than mice treated with immunoliposomes containing control siRNA. In addition, total cell number in tumors isolated from mice treated with immunoliposomes containing calumenin-directed siRNA is lower than mice treated with immunoliposomes containing control siRNA.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of diagnosing chemotherapeutic drug resistance in a neoplastic cell, comprising:
a) detecting a level of calumenin expressed in a neoplastic cell sample by contacting the cell sample with a probe specific for calumenin, and wherein the neoplastic cell sample is not obtained, or derived from, a cervix squamous cell carcinoma;
b) detecting a level of calumenin expressed in a non-resistant neoplastic cell control sample of the same tissue type as the neoplastic cell sample by contacting the cell sample with a calumenin-specific probe; and
c) comparing the level of expressed calumenin in the neoplastic cell sample to a level of expressed calumenin in the non-resistant neoplastic cell,
wherein chemotherapeutic drug-resistance is indicated in the neoplastic cell sample if the level of calumenin expressed in the neoplastic cell sample is greater than the level of calumenin expressed in the non-resistant neoplastic control cell sample.

2. The method of claim 1, wherein the detection steps comprise isolating a cytoplasmic sample from the neoplastic cell sample and the non-resistant neoplastic control cell sample.

3. The method of claim 1, wherein detecting the level of expressed calumenin in the cell samples comprises contacting the cell samples with a calumenin targeting agent selected from the group consisting of ligands, synthetic small molecules, nucleic acids, peptidomimetic compounds, inhibitors, peptides, proteins, and antibodies.

4. The method of claim 1, wherein the calumenin-targeting agent comprises an anti-calumenin antibody or a calumenin binding fragment thereof.

5. The method of claim 4, wherein the level of antibody bound to calumenin is detected by immunofluorescence, radiolabel, or chemiluminescence.

6. The method of claim 1, wherein the detecting steps comprise hybridizing a nucleic acid probe to a complementary calumenin mRNA.

7. The method of claim 6, wherein the nucleic acid probe is selected from the group consisting of RNA, DNA, RNA-DNA hybrids, and siRNA.

8. The method of claim 6, wherein the level of nucleic acid probe hybridized to calumenin mRNA is detected with a label selected from the group consisting of fluorophores, chemical dyes, radiolabels, chemiluminescent compounds, colorimetric enzymatic reactions, chemiluminescent enzymatic reactions, magnetic compounds, and paramagnetic compounds.

9. The method of claim 1, wherein the neoplastic control cell sample is selected from the group consisting of lung carcinoma, lung adenocarcinoma, colon carcinoma, ovarian carcinoma, and ovarian adenocarcinoma.

10. The method of claim 1, wherein the neoplastic cell sample to be tested is isolated from a mammal.

11. The method of claim 10, wherein the neoplastic cell sample to be tested is isolated from a human.

12. The method of claim 1, wherein the neoplastic cell sample to be tested comprises a breast adenocarcinoma.

13. The method of claim 1, wherein the potentially chemotherapeutic drug-resistant neoplastic cell sample is isolated from a tissue selected from the group consisting of breast, skin, lymphatic, prostate, bone, blood, brain, liver, thymus, kidney, lung, and ovary.

14. A method of treating a neoplasm in a patient in need thereof, comprising:
a) administering an effective amount of a calumenin-targeting agent to the patient, the targeting agent being capable of binding to calumenin expressed in the neoplasm; and
b) administering to the patient an effective amount of a chemotherapeutic drug,
wherein the calumenin targeting agent, when bound to the neoplasm, increases the sensitivity of the neoplasm to the chemotherapeutic drug, and wherein the neoplasm is not, or is not derived from, a cervix squamous cell carcinoma.

15. The method of claim 14, wherein the calumenin-targeting agent bound to the neoplasm is internalized into the neoplastic cell.

16. The method of claim 14, wherein the calumenin-targeting agent comprises a liposome.

17. The method of claim 16, wherein the liposome comprises a neoplastic cell-targeting agent on its surface.

18. The method of claim 14, wherein the calumenin-targeting agent is selected from the group consisting of ligands, nucleic acids, synthetic small molecules, peptidomimetic compounds, inhibitors, peptides, proteins, and antibodies.

19. The method of claim 18, wherein the calumenin-targeting agent comprises a nucleic acid.

20. The method of claim 19, wherein the nucleic acid is complementary to a calumenin mRNA.

21. The method of claim 19, wherein the nucleic acid is selected from the group consisting of RNA, DNA, RNA-DNA hybrids, and siRNA.

22. The method of claim 20, wherein the siRNA comprises 19 contiguous nucleotides of SEQ ID NO: 2.

23. The method of claim 20, wherein the siRNA comprises 25 contiguous nucleotides of SEQ ID NO: 4.

24. The method of claim 18, wherein the calumenin-targeting agent comprises an antibody or calumenin binding fragment thereof.

25. The method of claim 18, wherein the neoplastic cell-targeting agent comprises an antibody, or antigen-binding fragment thereof, specific for a cell marker selected from the group consisting of multidrug resistance protein 1, BRCP, p53, vimentin, α-enolase, nucleophosmin, and HSC70.

26. The method of claim 14, wherein the calumenin-targeting agent is administered to the patient by injection at the site of the neoplasm.

27. The method of claim 14, wherein the calumenin-targeting agent is administered to the patient by surgical introduction at the site of the neoplasm.

28. The method of claim 14, wherein the calumenin-targeting agent is administered to the patient by inhalation of an aerosol or vapor.

29. The method of claim 14, wherein the neoplasm to be treated is chemotherapeutic drug-resistant.

30. The method of claim 14, wherein the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristinee, Vinorelbine, and combinations thereof.

31. A kit for detecting chemotherapeutic drug resistance in a neoplastic cell sample, comprising:
a) a first probe for the detection of calumenin;
b) a second probe for the detection of chemotherapeutic drug resistance, the second probe being specific for a marker selected from the group consisting of multidrug resistance protein 1, BRCP, p53, vimentin, α-enolase, nucleophosmin, and HSC70; and
c) detection means for identifying probe binding to a target.

32. The kit of claim 31, wherein the first probe is selected from the group consisting of ligands, nucleic acids, synthetic small molecules, peptidomimetic compounds, inhibitors, peptides, proteins, and antibodies.

33. The kit of claim 32, wherein the first probe is a nucleic acid that is complementary to mRNA encoding calumenin.

34. The kit of claim 33, wherein the nucleic acid is selected from the group consisting of RNA, DNA, RNA-DNA hybrids, and siRNA.

35. The kit of claim 32, wherein the first probe is a calumenin-specific antibody or binding fragment thereof.

36. The kit of claim 31, wherein the second probe comprises a nucleic acid complementary to an mRNA encoding multidrug resistance protein 1, BRCP, p53, vimentin, α-enolase, nucleophosmin, or HSC70.

37. The kit of claim 36, wherein the nucleic acid probe is selected from the group consisting of RNA, DNA, RNA-DNA hybrids, and siRNA.

38. The kit of claim 31, wherein the second probe comprises an antibody or calumenin binding fragment thereof.

39. The kit of claim 31, wherein the detection means is selected from the group consisting of fluorophores, chemical dyes, radiolabels, chemiluminescent compounds, colorimetric enzymatic reactions, chemiluminescent enzymatic reactions, magnetic compounds, and paramagnetic compounds.

40. A pharmaceutical formulation for treating a neoplasm, comprising:
a) a calumenin-targeting component;
b) a chemotherapeutic drug; and
c) a pharmaceutically acceptable carrier.

41. The pharmaceutical formulation of claim 40, wherein the calumenin-specific targeting component is selected from the group consisting of ligands, nucleic acids, synthetic small molecules, peptidomimetic compounds, inhibitors, peptides, proteins, and antibodies.

42. The pharmaceutical formulation of claim 41, wherein the calumenin-targeting component is a nucleic acid.

43. The pharmaceutical formulation of claim 43, wherein the nucleic acid is selected from the group consisting of RNA, DNA, RNA-DNA hybrids, and siRNA.

44. The pharmaceutical formulation of claim 41, wherein the calumenin-targeting component is a siRNA.

45. The pharmaceutical formulation of claim 44, wherein the siRNA has a GC content of at least 40%.

46. The pharmaceutical formulation of claim 44, wherein the siRNA comprises 19 contiguous nucleotides of SEQ ID NO: 2.

47. The pharmaceutical formulation of claim 44, wherein the siRNA comprises 25 contiguous nucleotides of SEQ ID NO: 4.

48. The pharmaceutical formulation of claim 41, wherein the calumenin-targeting agent comprises an antibody or calumenin-binding fragment thereof.

49. The pharmaceutical formulation of claim 40, wherein the calumenin-targeting agent comprises a liposome.

50. The pharmaceutical formulation of claim 49, wherein the liposome comprises a neoplastic cell-targeting agent on its surface.

51. The pharmaceutical formulation of claim 50, wherein the neoplastic cell-targeting agent is an antibody, or binding fragment thereof.

52. The pharmaceutical formulation of claim 51, wherein the neoplastic cell-targeting agent binds to a neoplastic cell marker selected from the group consisting of multidrug resistance protein 1, BRCP, p53, vimentin, α-enolase, nucleophosmin, and HSC70.

53. The pharmaceutical formulation of claim 40, wherein the chemotherapeutic drug is selected from the group consisting of Actinomycin, Adriamycin, Altretamine, Asparaginase, Bleomycin, Busulfan, Capecitabine, Carboplatin, Carmustine, Chlorambucil, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastinee, Vincristine, Vinorelbine, and combinations thereof.


SEQUENCE LISTING (9 sequences)

SEQ ID NO: 1 - RNA, 19 nt, Artificial Sequence
  Description of Artificial Sequence Synthetic oligonucleotide
gaaggaccguguacaucau

SEQ ID NO: 2 - RNA, 19 nt, Artificial Sequence
  Description of Artificial Sequence Synthetic oligonucleotide
augauguacacgguccuuc

SEQ ID NO: 3 - RNA, 25 nt, Artificial Sequence
  Description of Artificial Sequence Synthetic oligonucleotide
gggugcugaagaagcaaagaccuuu

SEQ ID NO: 4 - RNA, 25 nt, Artificial Sequence
  Description of Artificial Sequence Synthetic oligonucleotide
aaaggucuuugcuucuucagcaccc

SEQ ID NO: 5 - RNA, 25 nt, Artificial Sequence
  Description of Artificial Sequence Synthetic oligonucleotide
ccaggguuccuaaucggauuugcua

SEQ ID NO: 6 - DNA, 21 bp, Artificial Sequence
  Description of Combined DNA/RNA Molecule Synthetic oligonucleotide
  Description of Artificial Sequence Synthetic oligonucleotide
gaaggaccguguacaucautt

SEQ ID NO: 7 - DNA, 21 bp, Artificial Sequence
  Description of Combined DNA/RNA Molecule Synthetic oligonucleotide
  Description of Artificial Sequence Synthetic oligonucleotide
augauguacacgguccuuctt

SEQ ID NO: 8 - RNA, 25 nt, Artificial Sequence
  Description of Artificial Sequence Synthetic oligonucleotide
acaaaugugaaugcagaccaaagaa

SEQ ID NO: 9 - RNA, 25 nt, Artificial Sequence
  Description of Artificial Sequence Synthetic oligonucleotide
uucuuuggucugcauucacauuugu